(19) United States
(12) Patent Application Publication
(10) Pub. No.: US 2026/0079153 A1
(43) Pub. Date: Mar. 19, 2026

(54) GENERATION OF MATURE NEURONS DIFFERENTIATED FROM PLURIPOTENT STEM CELLS

(71) Applicant: The Trustees Of Columbia University In The City Of New York, New York, NY (US)

(72) Inventors: Hynek Wichterle, New York, NY (US); Brian Jude Joseph, New York, NY (US); Chaolin Zhang, Scarsdale, NY (US)

(21) Appl. No.: 19/401,464

(22) Filed: Nov. 26, 2025

Related U.S. Application Data
(63) Continuation-in-part of application No. PCT/US2024/032048, filed on May 31, 2024.
(60) Provisional application No. 63/505,406, filed on May 31, 2023.
(60) Provisional application No. 63/645,759, filed on May 10, 2024.

(51) Int. Cl. G01N 33/50; C12N 5/0793; C12N 15/86

(52) U.S. Cl. CPC G01N 33/5091; C12N 5/0619; C12N 15/86; C12N 2506/45; C12N 2740/15043

(57) ABSTRACT

Methods for generating mature neurons differentiated from pluripotent stem cells, such as to facilitate the study of late-onset neurodegenerative disease, are disclosed. The methods include overexpressing the splicing factor muscleblind like splicing regulator 2 (Mbnl2) in cortical neurons. In some aspects, the method further comprises overexpressing RNA binding fox-1 homolog 1 (Rbfox1) and/or reducing expression of polypyrimidine tract binding proteins PTBP1/2. The methods also include accelerated derivation of mature neuron and generation of a tau pathology model. Also disclosed are constructs and compositions for accelerated derivation of mature neuron and/or generation of a tau pathology model.

### CROSS-REFERENCE TO RELATED APPLICATIONS

The present application is a Continuation-in-Part application of International Application No. PCT/US2024/032048, filed on May 31, 2024, which claims priority to U.S. Provisional Patent Application 63/505,406, filed May 31, 2023, and to U.S. Provisional Patent Application 63/645,759, filed May 10, 2024. The foregoing applications are hereby incorporated by reference in their entireties.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 38, 171 bytes XML file named “44010_160WO-PCT” created on May 31, 2024.

### TECHNICAL FIELD

The disclosure relates to a disease model based on alternative-splicing methods to produce mature neurons differentiated from pluripotent stem cells.

### BACKGROUND

Incurable aging-related neurodegenerative diseases are a growing public health crisis. The ability to generate substantial quantities of disease-pertinent neuronal types, with and without predisposing mutations, holds great promise for probing disease mechanisms and developing therapies. Traditionally, model organisms have been utilized to understand disease mechanisms and to develop therapies, but these models fail to reflect human-specific biology. The advent and development of stem cell technologies hold great promise for human neurodegenerative disease modelling and regenerative medicine. It is now possible to generate large quantities of neurons of diverse identities, providing valuable resources for the study of previously inaccessible cell types. However, while tremendous progress has made it possible to differentiate pluripotent stem cells to neurons of various developmental lineages, current protocols yield neurons that fail to mature in vitro and stall at an embryonic identity. This is a major bottleneck of modeling late-onset disease in the dish is that current protocols generate neurons of embryonic age, limiting the in-depth study of pathology that manifests only in adult neurons. Critically, this reflects a lack of understanding of the molecular mechanisms orchestrating neuronal maturation.

This clearly reflects a fundamental gap in knowledge concerning underlying molecular programs that drive neuronal maturation and aging and limits the potential of stem-cell-based interrogations of late-onset diseases. Accordingly, there is a need to develop technologies that can produce stem cell-derived mature neurons in vitro in order to bridge this fundamental gap.

### SUMMARY OF THE DISCLOSURE

Disclosed herein is a method of generating mature neurons (for example, mature motor neurons) from differentiated stem cells. In certain embodiments, the method comprises differentiating pluripotent stem cells into neurons; and overexpressing Mbnl2 in the differentiated pluripotent stem cells. In some aspects, Mbnl2 is overexpressed in the differentiated pluripotent stem cells by nucleofecting the differentiating pluripotent stem cell with a lentivirus construct for overexpressing Mbnl2. In some aspects, the lentivirus construct for overexpressing Mbnl2 has a sequence set forth in SEQ ID NO. 2 or SEQ ID NO. 3. In some embodiments, the pluripotent stem cells express Cre. In some implementations, the method further comprises overexpressing Rbfox1 in the pluripotent stem cells. In some embodiments, the method further comprises reducing expression of PTBP1/2 in the pluripotent stem cells.

In other embodiments of the method of generating mature neurons from pluripotent stem cells, the method comprises conditionally expressing Mbnl2 in the pluripotent stem cells to produce transfected stem cells; differentiating the transfected stem cells into neurons; and inducing expression of Mbnl2 in during the step of differentiating the transfected stem cells. In some aspects, Mbnl2 is conditionally expressed in the pluripotent stem cells by transfecting the pluripotent stem cell with a conditional expression construct for overexpressing Mbnl2. In some implementations, the method further comprises overexpressing Rbfox1 in the pluripotent stem cells. In some embodiments, the method further comprises reducing expression of PTBP1/2 in the pluripotent stem cells.

A method of accelerating maturation of a neuron (such as a motor neuron), is also disclosed. The method comprises overexpressing Mbnl2 in the neuron; overexpressing Rbfox1 in the neuron; and reducing expression of PTBP1/2 in the neuron. The method requires simultaneously modulating the expression of Mbnl2, Rbfox1, and PTBP1/2 in the neuron. In certain implementations, Mbnl2 is overexpressed in the neuron by nucleofecting the neuron with a lentivirus construct for overexpressing Mbnl2. In some aspects, the lentivirus construct for overexpressing Mbnl2 has a sequence set forth in SEQ ID NO. 2 or SEQ ID NO. 3. In some embodiments, the neuron expresses Cre. In other implementations, Mbnl2 is overexpressed in the pluripotent stem cells by transfecting the pluripotent stem cell with a conditional expression construct for overexpressing Mbnl2. Some embodiments of the method further comprises overexpressing Rbfox1 in the neuron and/or reducing expression of PTBP1/2 in the neuron. In particular implementations, the neuron is differentiated from a culture of pluripotent stem cells.

Compositions for inducing maturation of a neuron are also disclosed. In one embodiment, the composition comprises a construct for overexpressing Mbnl2 and a construct for reducing expression of PTBP1/2. In another embodiment, the composition comprises a construct for overexpressing Mbnl2 and Rbfox1 and a construct for reducing expression of PTBP1/2.

The disclosure further includes a method of generating a tau pathology model. The method comprises overexpressing Mbnl2 in the neuron; overexpressing Rbfox1 in the neuron; and reducing expression of PTBP1/2 in the neuron. The method requires simultaneously modulating the expression of Mbnl2, Rbfox1, and PTBP1/2 in the neuron. In some implementations, the neuron is a motor neuron.

The foregoing and other aspects, features, and advantages will be apparent from the DESCRIPTION and DRAWINGS, and from the CLAIMS if any are included.

### BRIEF DESCRIPTION OF THE DRAWINGS

Implementations will hereinafter be described in conjunction with the appended and/or included DRAWINGS, where like designations denote like elements.

The patent or application file contains at least one drawing executed in color. Copies of this patent or patent application publication with color drawing(s) will be provided by the Office upon request and payment of the necessary fee.

FIG. 1 is a diagram representation of the stages needed for a stem cell to differentiate into a mature neuron, including potentially unknown splicing-regulatory programs that might provide an important system for mechanistic studies related to ageing, late disease modeling and drug screening targeting the nervous system.

FIG. 2A is a toy diagram of cassette exon alternative splicing (AS) that results in skipping or inclusion of exon 2 (E2) in mRNA. FIG. 2B shows bar graph showing Mbnl2 expression increases during development.

FIG. 3 is an image depicting rt-PCR of E10 in controls and MAPT 10+16 (Patient1,2) neurons at day 100 of differentiation. Controls do not activate E10 inclusion, 10+16 induces E10 far less than normal human brain levels (+ve). GAPDH as control is shown below.

FIG. 4A is a graph of splicing profiles of various samples at different maturation stages are projected into 2D by PCA with maturation stages predicted using Splicescope and mouse embryonic stem cell (mESC)-derived MNs are labelled according to in vitro age (D1, D5 and D10) for which each dot represents a unique neuronal dataset and key developmental points are highlighted. Two time series of maturing ESC-derived motor neurons (MN) and in vivo motor neurons are highlighted. FIG. 4B depicts heatmaps showing comparison of splicing profiles of mouse cortex tissues and ESC-derived motor neurons at different maturation stages. Two groups are highlighted: early switch exons which change patterns immediately after E10.5 and late switch exons which change patterns postnatally and mESC-derived spinal MNs that fail to activate early and late switching patterns in the dish at different timepoints (heatmap on the right).

FIGS. 5A-5E demonstrate that Mbnl is a master regulator of splicing switches during neuronal maturation a heatmap of developmental splicing and Mbnl2 targets. As shown in FIG. 5A, postnatal AS switch exons are broadly regulated by Mbnl2. Cassette exons with late developmental splicing switches conserved between mouse and human brains are shown on the left. Regulation of these exons by Mbnl, as determined by comparison of wild type (WT) vs. Mbnl1/2 double knockout (DKO) brains in mice as well as control and myotonic dystrophy type 1 (DM1) brains in humans is shown on the right (red bar: splicing activation). The proportion of exons regulated by Mbnl is indicated below the heatmap. (pcw: post-conception weeks). FIG. 5B is a schematic showing regulation of MAPT exon 10 by Mbnl. FIG. 5C is a graph depicting the changes of MBNL2 mRNA expression level (left axis) and MAPT exon 10 inclusion (right axis) in developing human cortices (left panel) as quantified by RNA-seq with Exon 10 inclusion in control and DM1 patient brains is also shown (right panel). FIG. 5D is a similar graph as FIG. 5C but for mouse brain. Mbnl expression drives exon 10 inclusion in human and mouse MAPT minigenes. FIG. 5E shows that Mbnl expression drives exon 10 inclusion in human and mouse MAPT minigenes. Exon 10 inclusion in HEK293T cells transfected with empty pcDNA vector, cells with Mbnl1/2 knockdown using siRNAs (siMBNL1/2) and cells with overexpression of FLAG-Mbnl2 (pFLAG-MBNL2). Representative images (E) show the confirmation of protein expression using immunoblots (control: GAPDH) and exon 10 inclusion using RT-PCR. Quantification of exon 10 inclusion (mean±SEM from n=3 replicates) is shown at the bottom.

FIG. 6 is a schematic of the strategy to generate iMbnl2 mouse ESCs. Inducible Cassette Exchange (ICE) locus and cassette exchange to introduce gene of interest and drive G418 resistance representing the strategy to generate iMbnl2 mouse ESCs. The ICE locus originally consists of inducible Cre, and a non-functional neomycin resistance cassette (Aneo). Upon cassette exchange with the Mbnl2 donor plasmid, the Cre is replaced with a Mbnl2 and Aneo is repaired, allowing selection of ESCs with the desired recombination event.

FIG. 7 is a diagram showing differentiation of the iMbnl2 mESC line in the presence of retinoic acid and sonic hedgehog agonist (SAG). By Day 5, the treatment leads to formation of embryoid bodies (EBs) with ˜30% MNs (GFP+). EBs will be dissociated, MNs sorted with GFP and cultured for the indicated time for phenotypic and transcriptomic readouts. Mbnl2 expression is induced in progenitor MNs by adding doxycycline on late Day 3, and SAT treatment continues during the course of the experiment.

FIG. 8 shows fluorescent microscopy indicating robust induction of Mbnl2 in Day 5 EBs and plated MNs at DIV1.

FIG. 9 shows the design of Mbnl2 construct excluding viral elements (top) and the infection and validation of inducible expression in two cell lines (bottom). TRE: Tet response element; PGK: PGK-1 promoter; rtTA: reverse tetracycline-controlled transactivator; and WPRE: Woodchuck Hepatitis Virus (WHV) post-transcriptional regulatory element.

FIGS. 10A and 10B depict the strategy to drive 4R tau expression in human neurons with Mbnl2. FIG. 10A shows the strategy to express Mbnl2 in nascent neurons and sustain Mbnl2 during cultures. Wildtype and 10+16 cells will be collected on day 45 to assess MAPT splicing and tau isoforms. For ASO screening, oligos will be transfected on day 39 and cells collected on day 45. For tau burden assays, tau seeds will be supplied on day 42 and readouts performed on day 45. ASOs+tau seeding combined, as indicated. FIG. 10B is a diagraph representing MAPT exon 10 (E1) and flanking region. Mbnl2 binding sites 1 and 2 are shown along with MBNL2 CLIP tags. These regions will be tiled with ASOs to screen for exon 10 regulating oligos.

FIGS. 11A-11K demonstrate Mbln2 can accelerate mouse MN transcriptomic maturation. FIG. 11A shows Mbnl2 activates about 40% of missing developmental switches in MNs 15 days in vitro (DIV15), but FIG. 11B shows that Mbnl2 activation does not affect development gene expression. The design of an exemplary FLEx-Mbnl2 lentivirus used for Cre-dependent overexpression of Mbnl2 is shown FIG. 11C. FIG. 11D is a representative western blot demonstrating that the FLEx-Mbnl2 construct yields Mbnl2 protein with co-expression of CRE but not control (GFP). HEK293T cells were transfected with FLEx-Mbnl2 and Cre plasmids (lane 1) or FLEx-Mbnl2 and GFP plasmids (lane 2). FIG. 11E depicts schematics of the experimental setup culturing motor neurons with FLEx-Mbnl2 lentivirus using mouse ESCs (top) and human induced pluripotent stem cells (iPSCs) (bottom) bearing a Chat driven Cre in post-mitotic MNs is also represented. FIG. 11F shows a representative immunostaining of ectopic Mbnl2 in stem cell-derived motor neurons. The red arrow indicates Mbnl2 overexpression using FLEx-Mbnl2, and the white arrow indicates endogenous Mbnl2 expression. Dissociated MNs were infected with FLEx-Mbnl2 or control virus and cultured for 14 days in vitro prior to immunostaining. FIG. 11G show that maturation dependent splicing events are activated in stem cell-derived motor neurons upon Mbnl2 overexpression by DIV 7. 4R, 2N, 1N, Ex and In are maturation-dependent splice isoforms. Mbnl2-neurons from mouse ESCs display accelerated electrophysiological maturation as show in patch-clamp electrophysiology experiments (FIG. 11H) and multi-electrode arrays (MFA) (FIG. 11I). In human iPSCs Mbnl2 expression overcomes traditional challenges inducing 4R tau in patient lines. As shown in FIG. 11J, stem cell-derived neurons do not express 4R tau. However, Mbnl2 expression in stem cell-derived motor neurons induces 4R tau expression and thus produces tauopathy cell lines (FIG. 11K).

FIG. 12A shows the experimental strategy to identify key periods when Mbnl2 activity is required to set the mature splicing pattern. Mbnl2 is either induced for a short period during motor neuron birth (Early Pulse), continuously (Continuous), or briefly during middle and late stages of culture (Mid. Pulse and Late Pulse). These conditions distinguish whether Mbnl2 is required briefly early on, continuously, or can be induced anytime in culture to drive splicing maturation in vitro. FLAG immunostaining (shown in FIG. 12B) show that ectopic Mbnl2 are detected in EBs containing motor neurons on day 6 upon completion of motor neuron differentiation.

FIGS. 13A and 13B represent the morphological and electrophysiological maturation of human ESC-derived motor neurons. As shown in FIG. 13A, human motor neurons increase in soma size and display neurite outgrowth in vitro over time. Representative images and MetaMorph tracking of soma and neural processes are shown on the left portion of FIG. 13A, and quantifications are shown on the right portion. FIG. 13B shows that human motor neurons in vitro show increase action potential firing, decrease in input resistance, and decrease in threshold potential in maturing cells. Representative traces are shown on the left of FIG. 13B, and quantifications are shown on the right.

FIGS. 14A-14D demonstrate combinatorial control of developmental splicing switches during neuronal maturation. FIG. 14A is a heatmap showing the dynamic expression of four families of tissue-specific RBPs, including Ptbp1/2, Nova1/2, Rbfox1-3 and Mbnl1/2. FIG. 14B shows that RBPs either antagonize (Ptbp2) or facilitate (Nova, Rbfox and Mbnl) the mature splicing pattern through activation or repression of exon inclusion. Splicing of Gabrg2 exon 9 in the cortex at different time points is represented in FIG. 14C. As shown in FIG. 14D, inclusion of Gabrg2 exon 9 is repressed by Ptbp and then activated by Nova, Rbfox and Mbnl during neuronal maturation. Validation of regulation by Nova, Rbfox and Mbnl suggests a synergistic effect on splicing activation.

FIGS. 15A-15C disclose a method of accelerating mouse MN transcriptomic maturation with Mbnl2 through combinatorial RBP perturbation. FIG. 15A depicts the design of FLEx-Mbnl2-t2A-Rbfox1 transgene that does not express RBPs under default configuration but robustly expresses Mbnl2 and Rbfox1 in Cre expressing cells. FIG. 15B depicts the design of pSico-Ptbp1/2 for conditional knockdown of Ptbp1/2. Under default configuration, the construct does not produce Ptbp1/2 targeting shRNAs, instead, it produces GFP. In Cre expressing cells, GFP is flipped out, and Ptbp1/2 shRNAs are produced. One shRNA construct will be used for Ptbp1 and another for Ptbp2. FIG. 15C depicts the schematic of experimental setup culturing motor neurons with FLEX-Mbnl2 lentivirus using mouse ESCs bearing a Chat driven Cre in post-mitotic MNs. Dissociated MNs are infected with FLEx-Mbnl2 or control virus and cultured for 14 days in vitro. Cells are profiled after fixed periods in culture to understand transcriptomic maturation.

FIGS. 16A-16C show the maturation of human motor neurons with RBP perturbation assays. FIG. 16A shows experimental strategy to perturb RBPs in human MN. Vacht-Cre MN is dissociated and infected with different individual or pooled lentivirus cocktails. Cells are profiled after fixed periods in culture to understand transcriptomic and functional maturation. FIG. 16B depicts immunostaining of ectopic Mbnl2 in human iPSC-derived motor neurons as visualized with fluorescent microscopy. MBNL2 is shown in green, while the neuron cell is shown in red. FIG. 16C shows that maturation-dependent splicing of exon 10 in the MAPT gene is activated in human motor neurons upon Mbnl2 overexpression. The 4R is a maturation dependent splice isoform.

FIGS. 17A-C is a representation of modeling ALS/TDP43 pathology in matured, iPSC-derived motor neurons is represented. PiggyBAC transposon for inducible Cre expression. The construct consists of the Cre drive by a Tet-promoter (TRE), and a CAGGs driven Tet activator (Tet-On 3G). The construct also contains a puromycin resistance cassette driven by the EF-1α promoter. Strategy to mature motor neurons derived from wildtype or ALS mutation-bearing iPSCs is also shown (B). iPSCs will first be nucleofected with a Cre piggyBAC construct (A) and transposase plasmid to stable insert inducible Cre locus into iPSCs. Stable integrant cells will be selected by puromycin treatment. Thereafter, iPSCs are differentiated into motor neurons in embryoid bodies (EBs). Cre will be induced in cells starting on Day 10 and EBs will be dissociated and plated on Laminin on D11 and differentiated for the next five days as adherent cells. During plating on Day 11, cells will be infected with a pooled cocktail of lentiviruses to drive mature splicing patterns. Proteinopathies induced in ALS motor neurons are shown via immunostaining (C). iPSC-derived motor neurons exposed to puromycin form stress granules containing TDP43 and G3BP1 that become stabilized in lines carrying ALS causing mutations in TDP43.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, an embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As used herein, the term “Mbnl” refers to the muscleblind like splicing regulator genes, which contains two forms: Mbnl1 and Mbnl2. In following typical naming conventions, the protein product of the muscleblind like splicing regulator genes is referred to herein as “MBNL1” or “MBNL2.”

As used herein, the term “Rbfox1” refers to the RNA binding fox-1 homolog 1 gene. In following typical naming conventions, the protein product of the RNA binding fox-1 homolog 1 gene is referred to herein as “RBFOX1.”

As used herein, the term “Ptbp” refers to the polypyrimidine tract binding protein genes, which contains two forms: Ptbp1 and Ptbp2. In following typical naming conventions, the protein product is referred to herein as “PTBP1” or “PTBP2.” “PTBP1/2” is utilized to refer to the entire family of pre-mRNA binding protein.

As used herein, the term “MAPT” refers to the microtubule associated protein tau gene whose transcript undergoes complex, regulated alternative splicing, to give rise to several mRNA species producing various versions/isoforms of the tau protein.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, an embodiment includes from the one particular value to the other particular value. Similarly, when values are expressed as approximations, by use of the antecedent “about,” it will be understood that the particular value forms another embodiment. All ranges are inclusive and combinable.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The development of a nervous system is a complex process that starts with the specification of neuronal identity and ends with a fine-tuned functional neuronal circuit. While neurons are generated during a brief embryonic window, maturation of the nervous system is a protracted process, lasting over weeks in mice and years in humans. During this time, young neurons undergo a complex process of migration, neurite outgrowth, synapse formation, electrophysiological maturation, neurite pruning, and modulation of synaptic strength, through which they are transformed into highly specialized information processing nodes within complex neural circuits. Although molecular and cellular changes accompanying neuronal maturation are being intensely investigated, mechanisms controlling acquisition of a mature neuronal state remain poorly understood. Thus, slow and incomplete neuronal maturation is a major impediment to effective modeling of the nervous system.

Technological advances in stem cell engineering enable direct differentiation of pluripotent stem cells (PSCs), including both embryonic stem cells (ESCs) and induced PSCs (iPSCs), into a growing number of well-defined neuronal cell types. These technologies have prompted an ever-increasing use of these cells for studying neurological diseases. However, the relatively immature state of stem cell-derived neurons remains a major impediment, particularly for effective modeling late-onset diseases (e.g. Alzheimer's disease, Parkinson's disease, and ALS) that affect only fully mature neurons. Efforts have also been made to make neurons by direct transdifferentiation from fibroblast, without reprogramming into PSCs. Interestingly, these neurons preserve the mature identity. However, the caveat of this transdifferentiation approach is that it is difficult to scale due to the scarcity of tissue sources and the limited cell number that can be produced; in addition, genetic manipulations of these cells are also challenging. Thus, discovering regulators that can facilitate the maturation of stem cell-derived neurons would have a profound impact on researching aging and late-onset neurological diseases that are becoming a major concern for the aging world population.

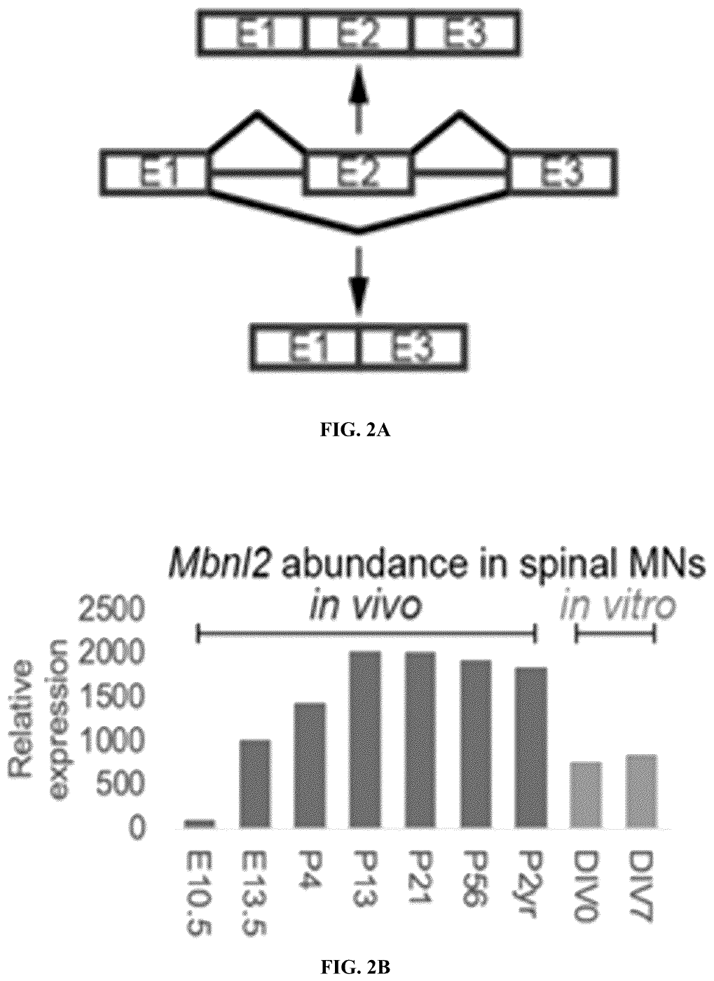

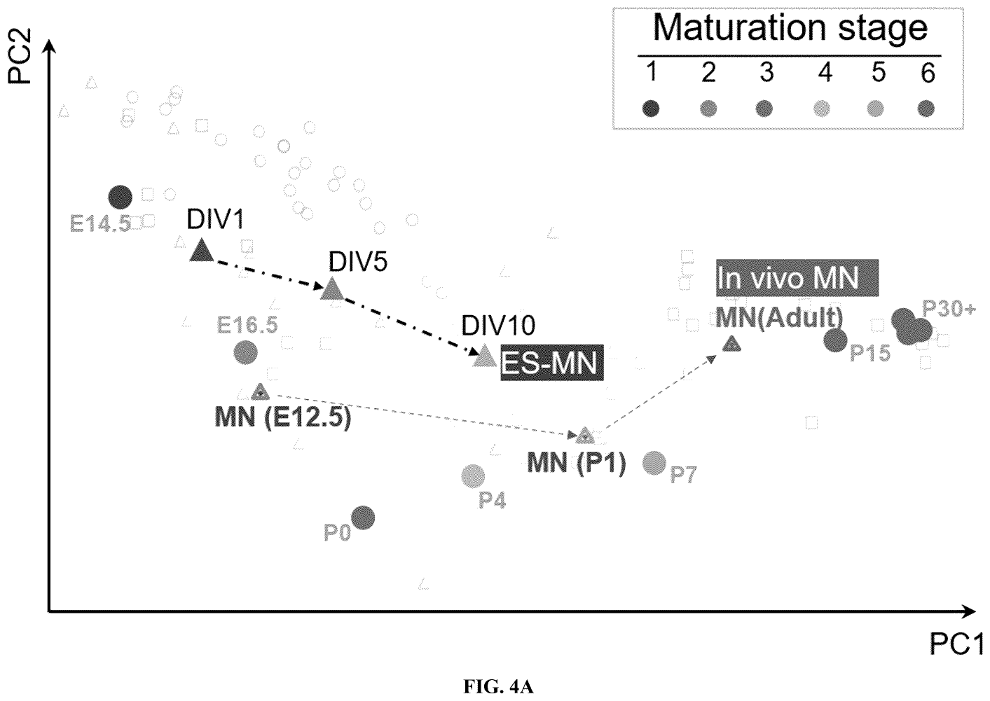

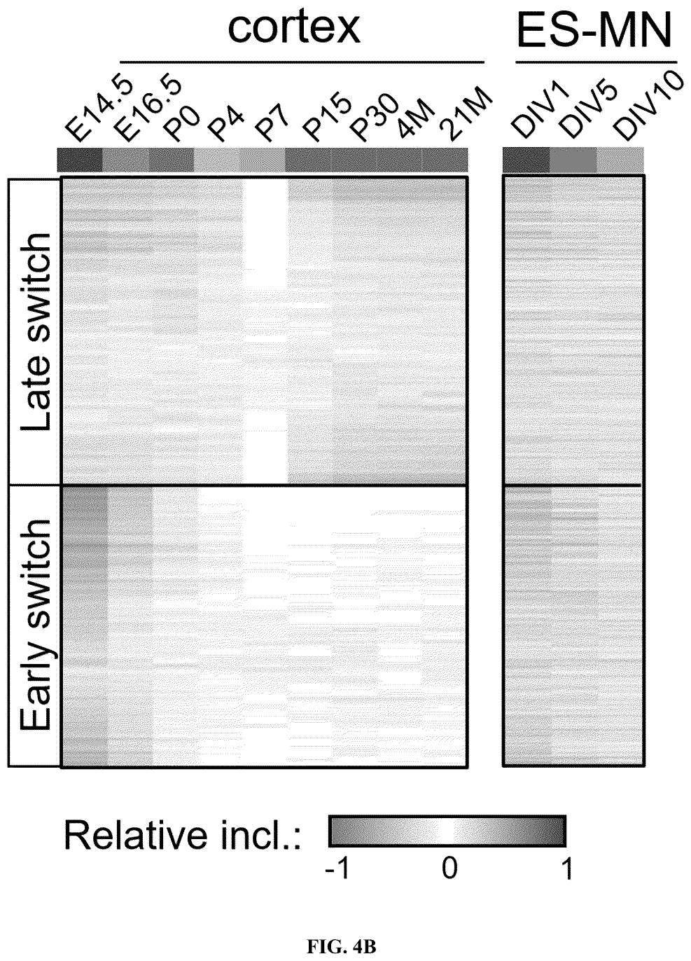

Alternative splicing (AS) is a mechanism in which multiple transcript and protein variants are generated from a single host gene. The mammalian nervous system employs AS to massively expand the complexity of genetic information encoded in the genome. Temporal examination of in vivo and differentiated neurons have revealed AS programs that are activated at distinct stages of neuronal development. Each program consists of hundreds of exons with highly correlated splicing patterns, suggesting broad regulatory capacity. Thousands of alternative exons show dramatic splicing switches at precise time points during neuronal maturation. These switches led to coordinated activation of adult transcript and protein isoforms involved in morphological and functional changes of neurons. Multiple RNA-binding protein (RBP) splicing factors, whose expression is developmentally regulated to drive the coordinated maturation-dependent splicing changes, have been identified. Thus, AS alone can be used to determine neuronal maturation stage, and gene enrichment analyses provide clear links connecting specific AS programs and stage-specific neurodevelopmental requirements (FIGS. 2A and 4B). While there are examples of individual AS exons with important roles in neurons, the functional significance of stage-specific AS programs is poorly studied and represents an important step towards understanding neurodevelopment. Regardless, engineering AS programs by manipulating the expression of key splicing factors in nascent neurons can be sufficient to overcome gene regulatory barriers and accelerate neuronal maturation at the molecular and functional levels, in a similar way that ectopic expression of Yamanaka factors reprograms somatic cells to pluripotent stem cells.

As shown in the Examples, exploiting endogenous splicing-regulatory mechanisms during neuronal maturation in vivo facilitates and accelerates the acquisition of mature neuron identity. As such, disclosed herein is a platform technology that revolutionizes mechanistic studies of neuronal maturation and aging, modeling of late-onset neurodegenerative diseases, and preclinical drug screening.

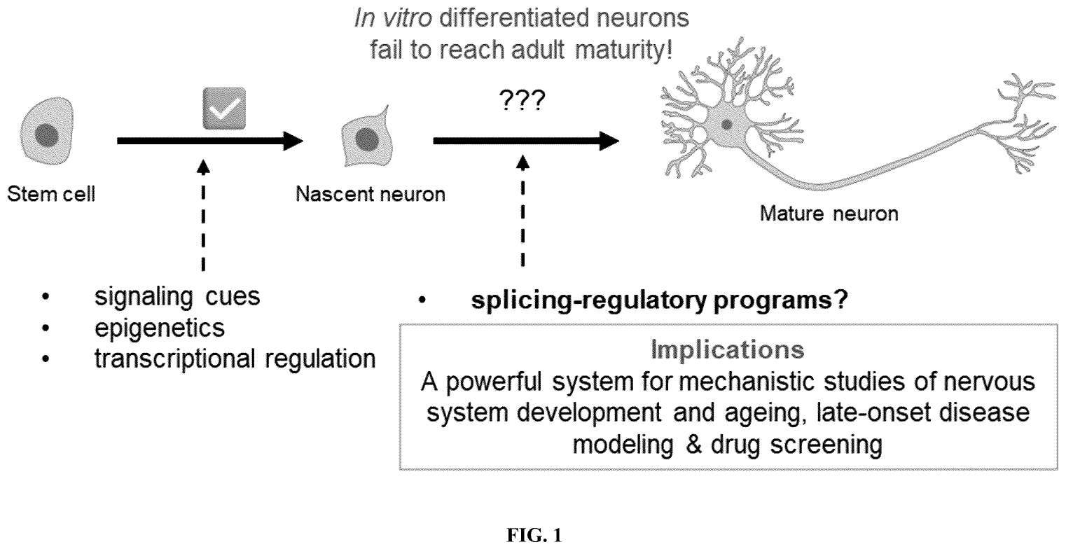

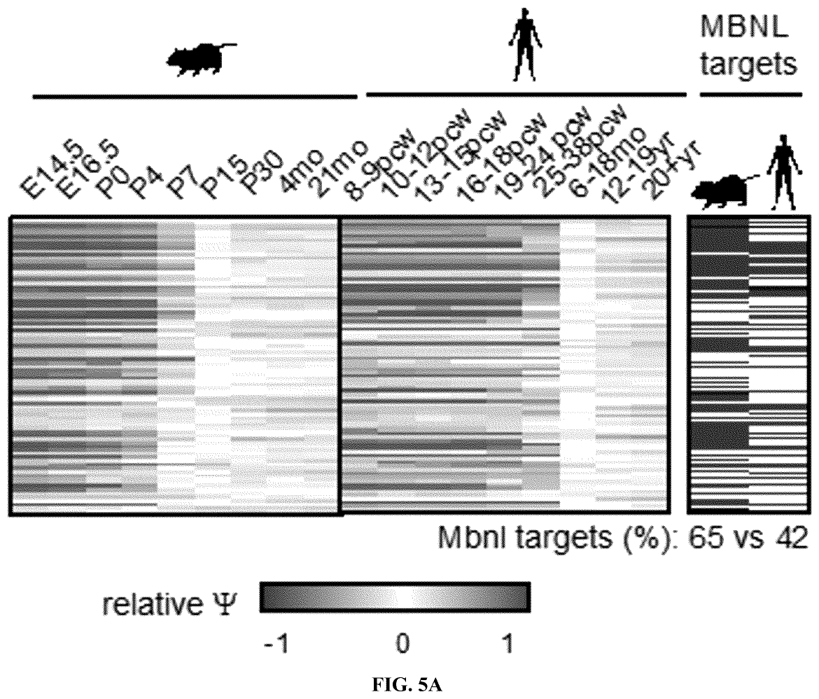

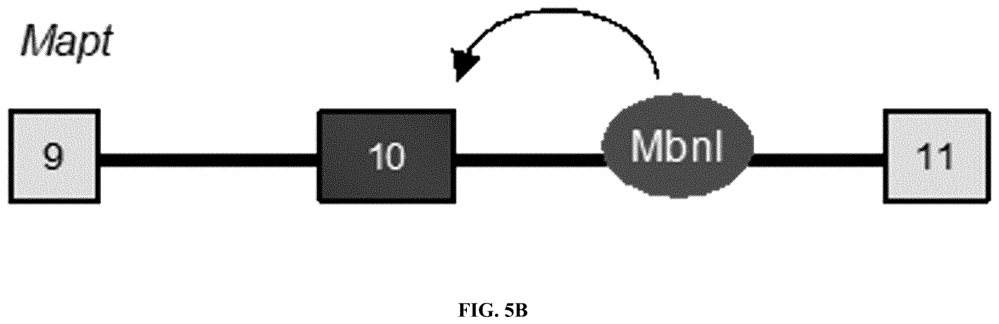

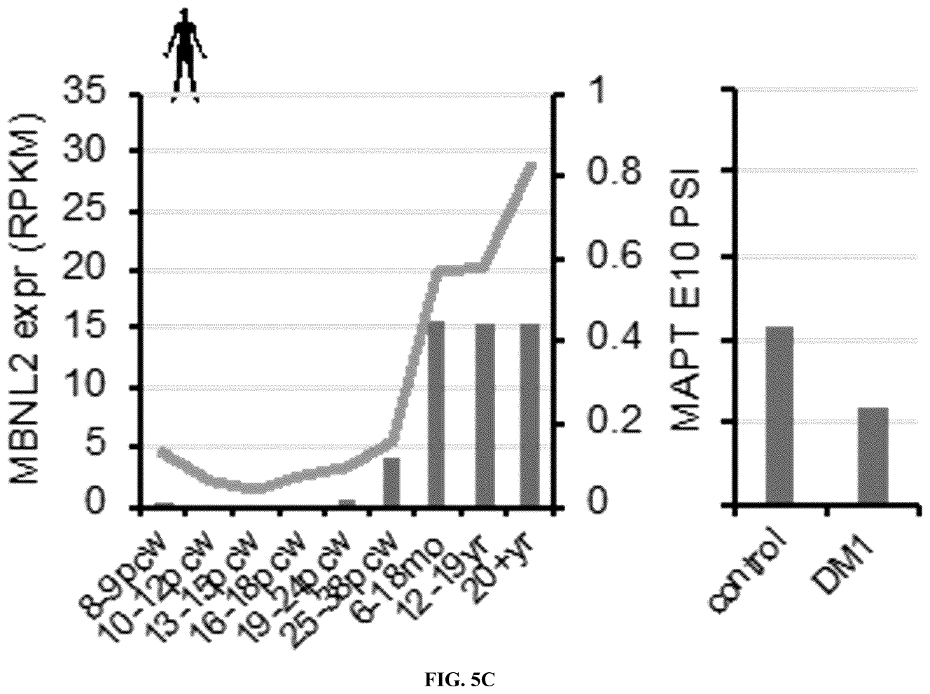

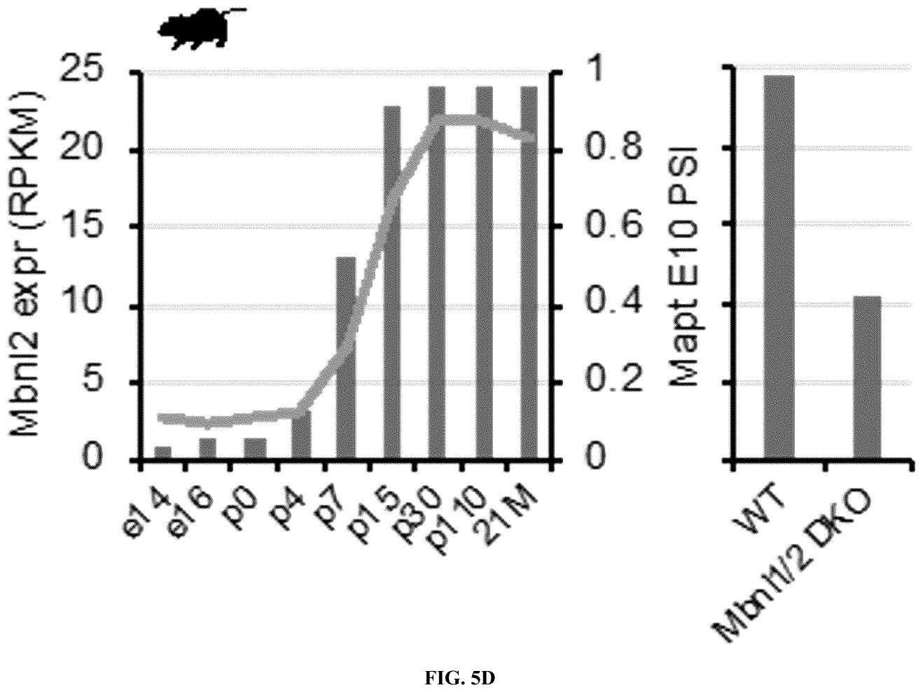

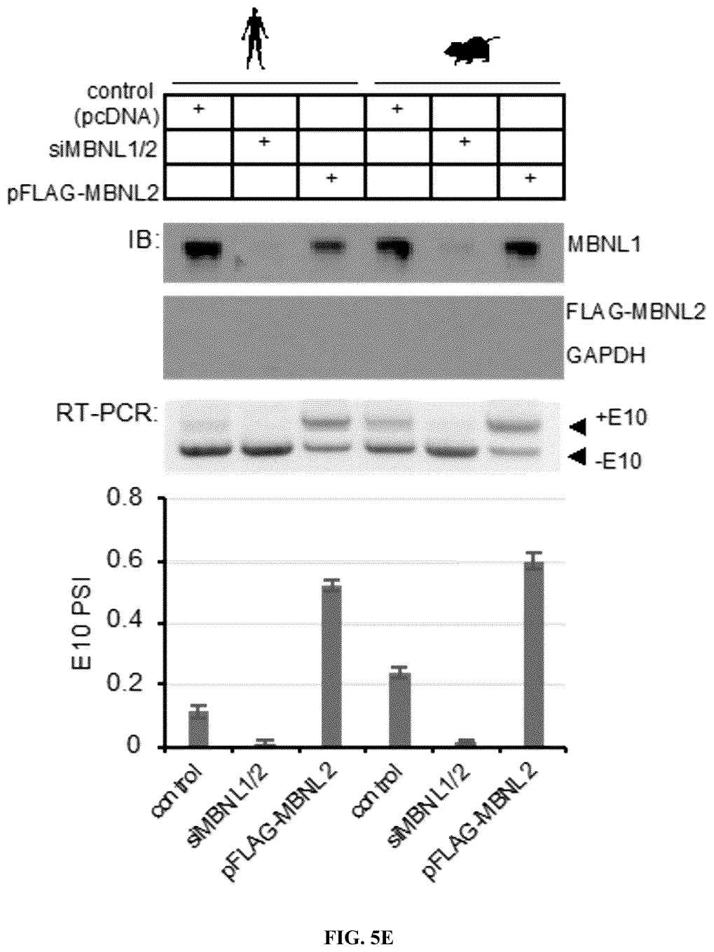

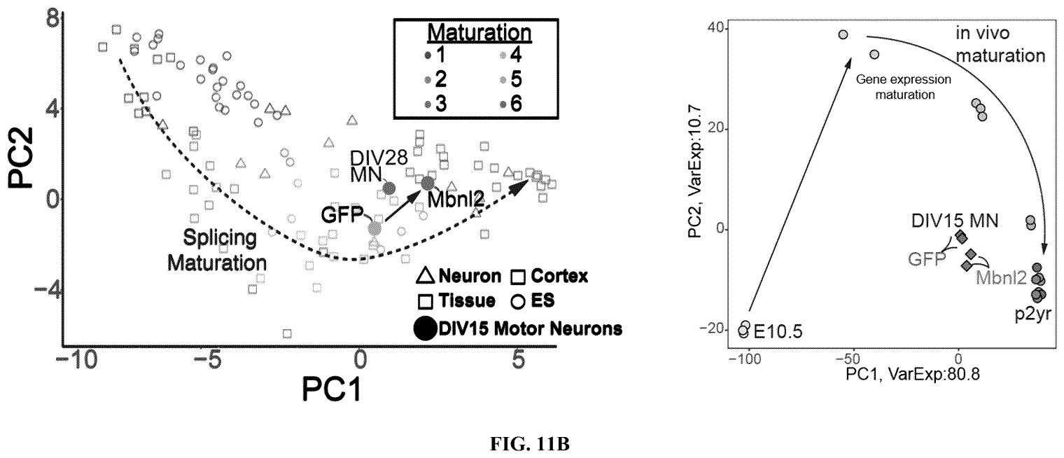

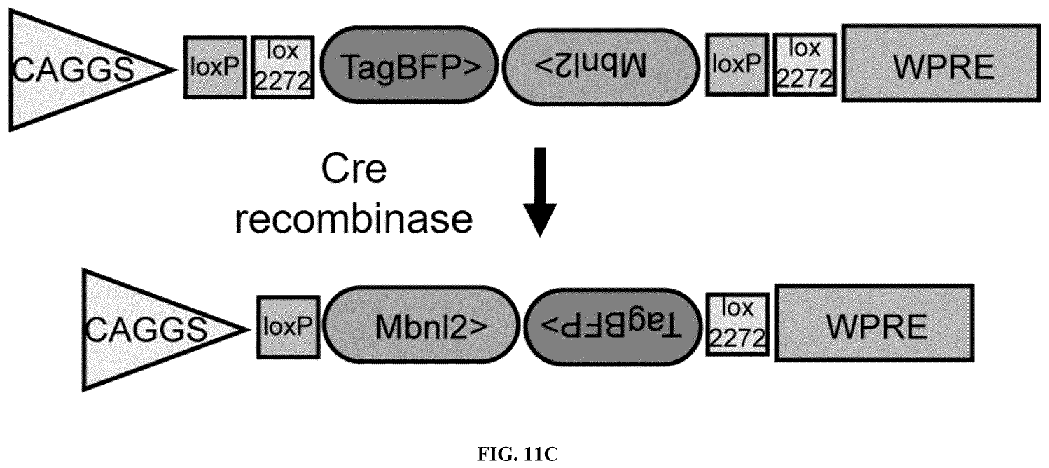

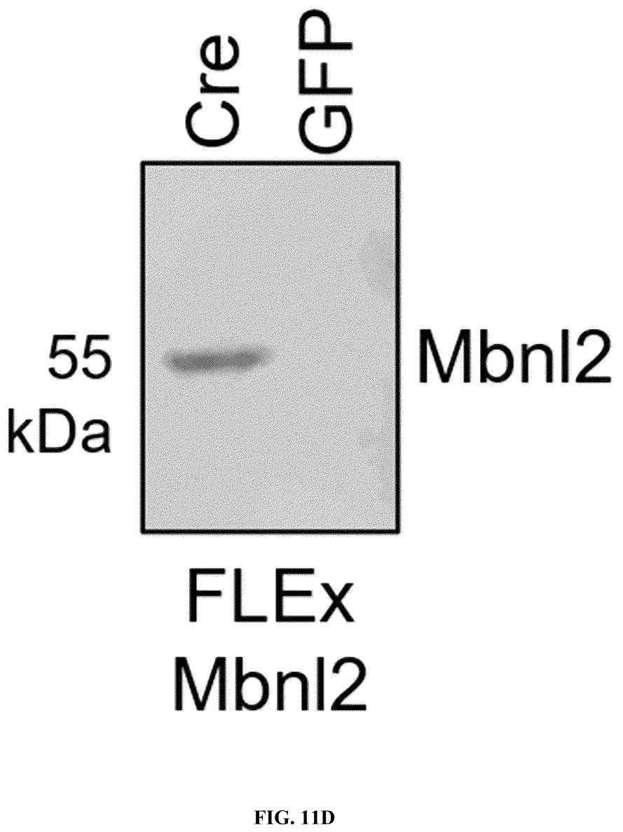

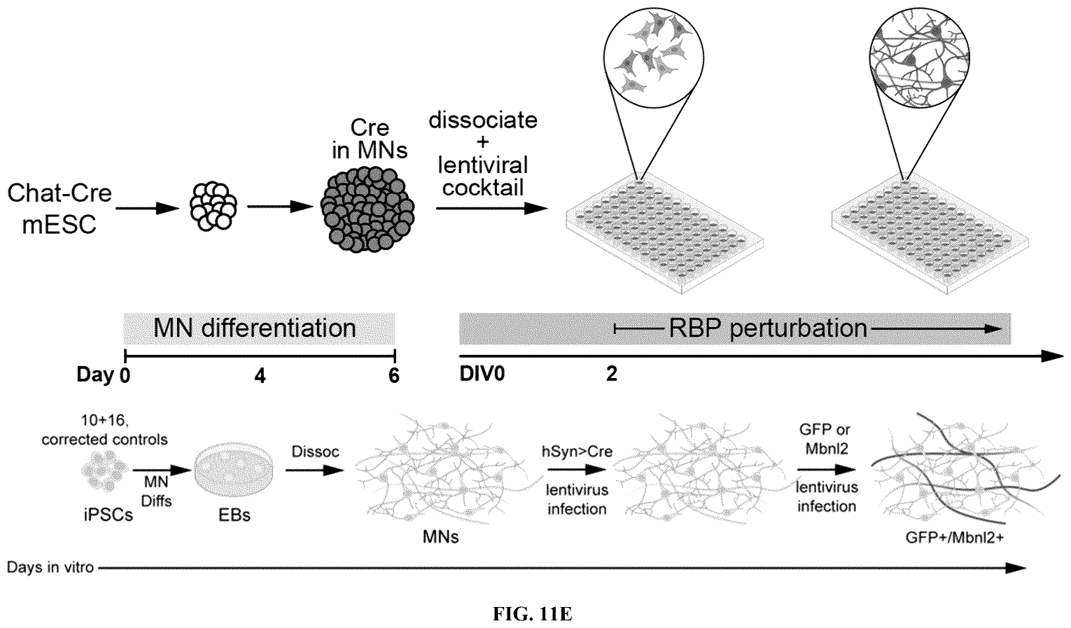

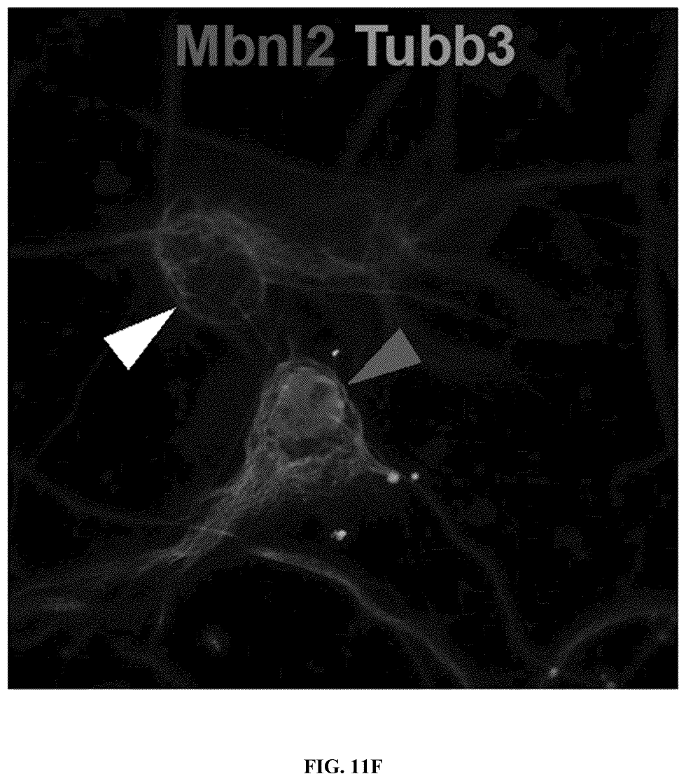

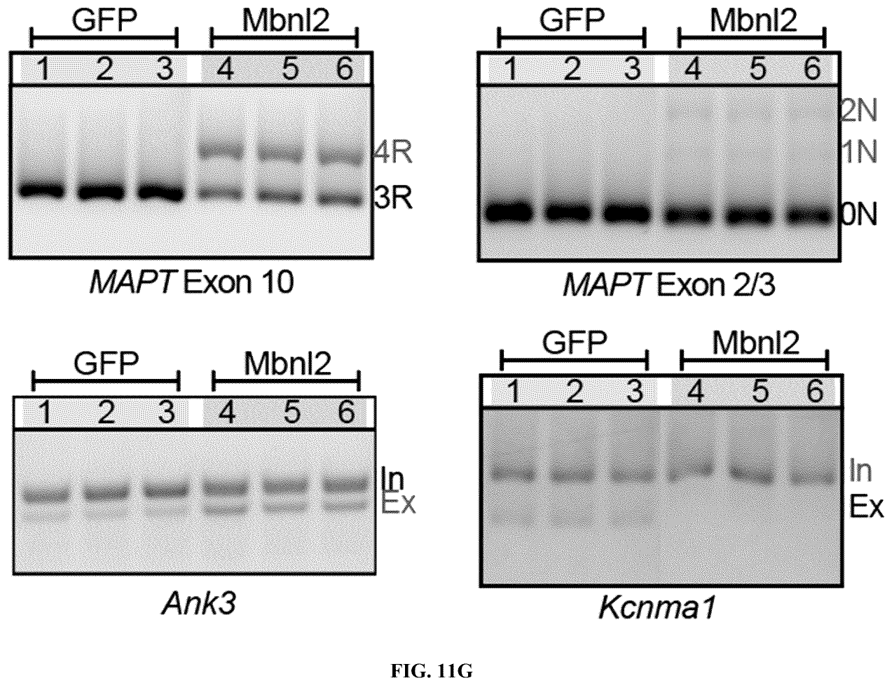

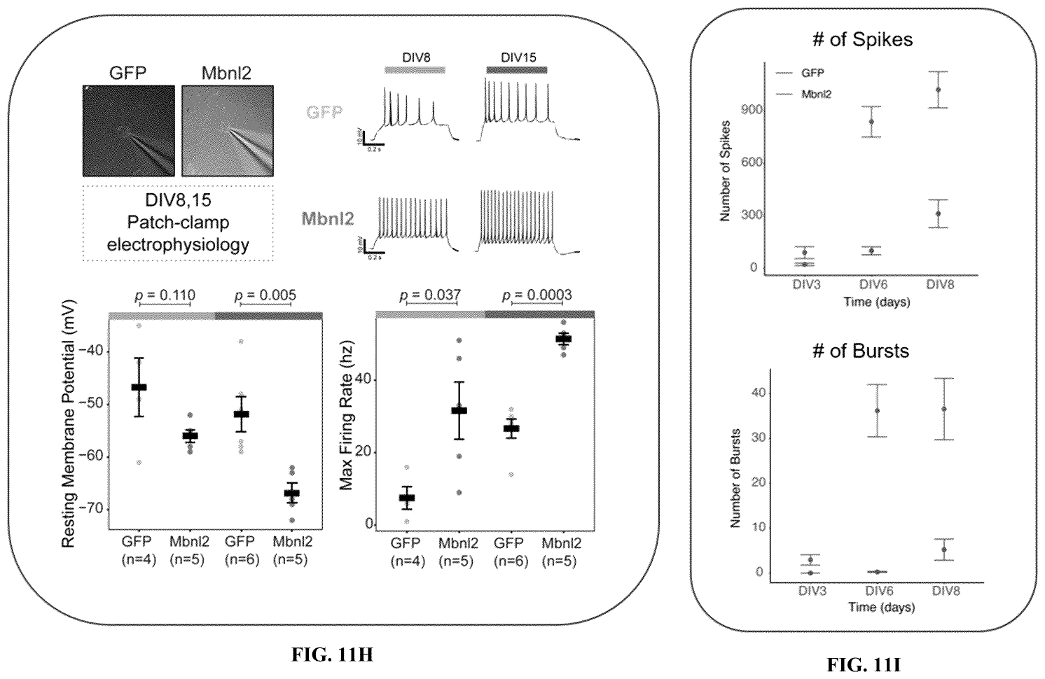

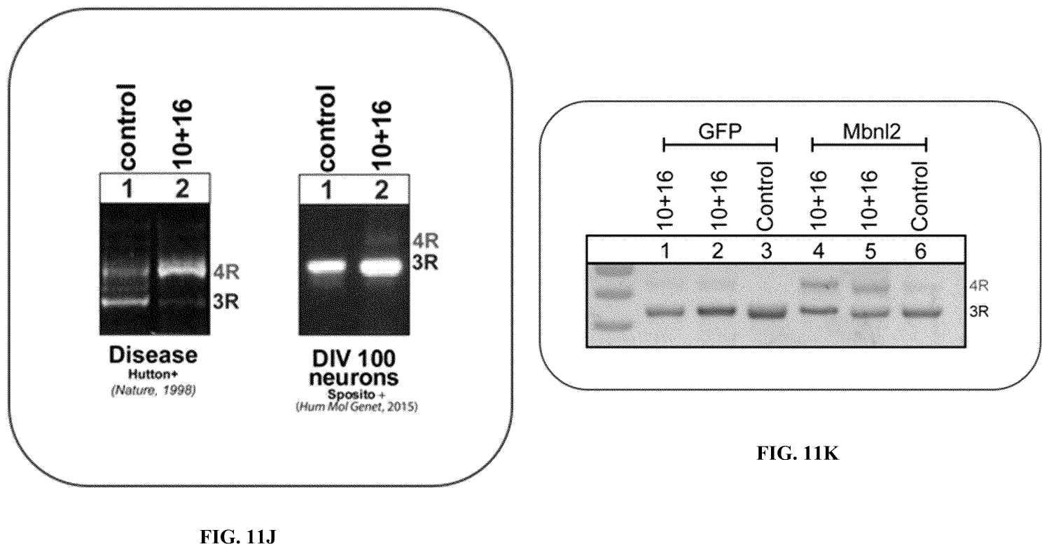

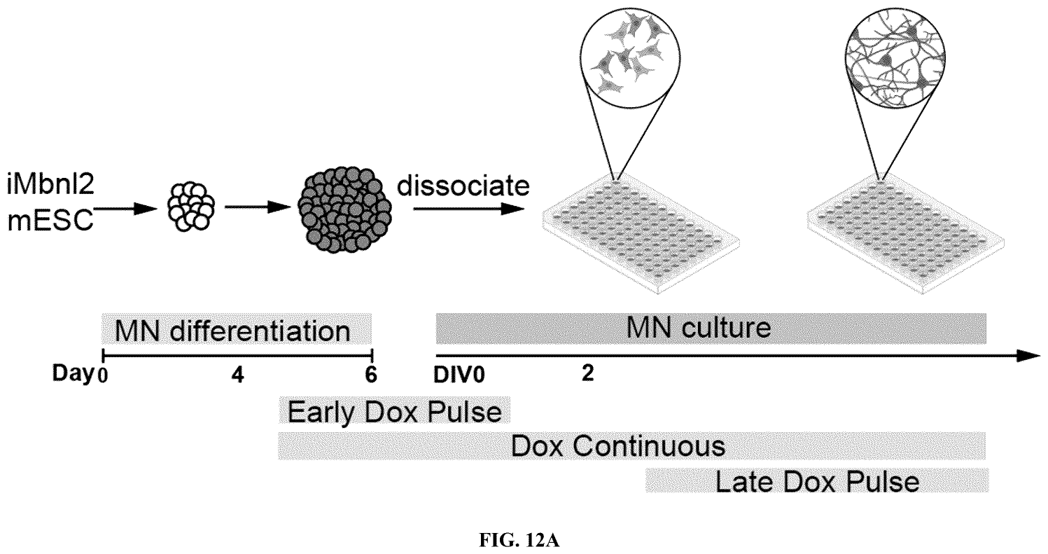

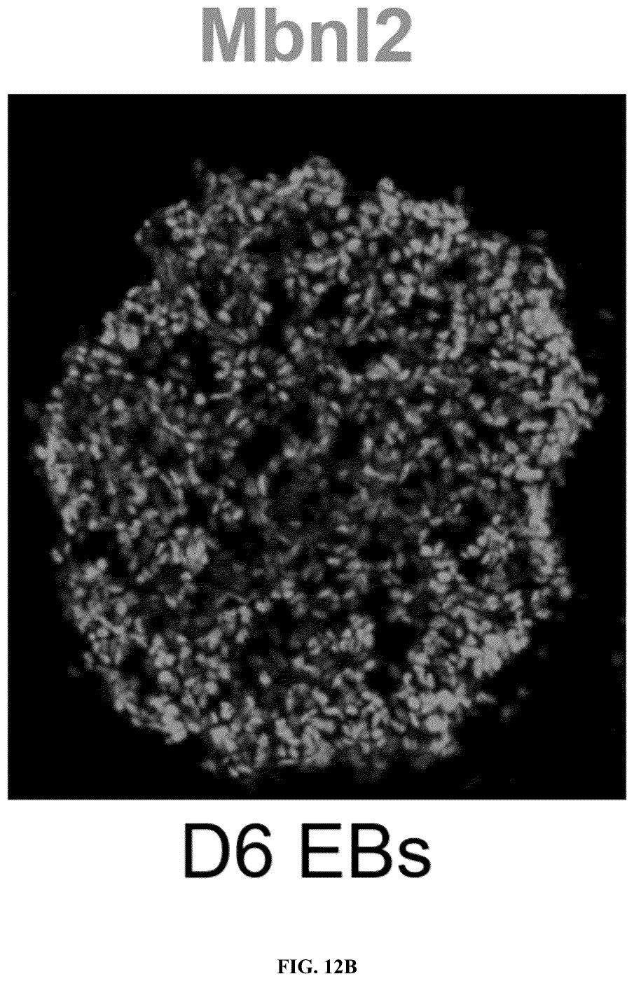

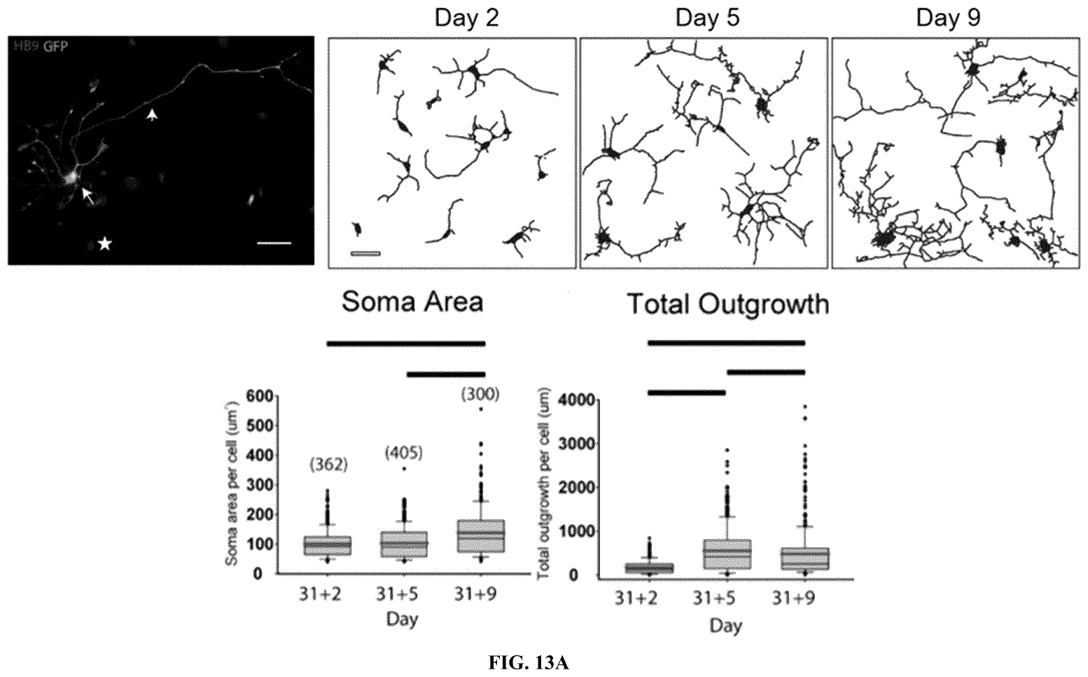

AS programs are executed by the activity of RNA binding proteins (RBPs) that directly bind pre-mRNAs and alter splice site choice. Neuron-specific or enriched RBPs orchestrate neuronal AS, including Nova, Rbfox, Ptbp2, nElavl, nSR100 and Mbnl2. These RBPs have been perturbed to directly examine the importance of neural AS programs, and depletion of RBPs in ES-derived neurons and the mouse brain confers strong impacts on physiological and transcriptomic maturation. In particular, Mbnl proteins regulates up to ⅔ of exons showing splicing switches during maturation (FIG. 5A) and Mbnl2 depletion or sequestration results in embryonic-like splicing patterns in neurons and patient brains with the multisystemic disease, myotonic dystrophy (DM). Mbnl2 activates ˜40% of missing developmental switches in DIV15 MNs but does not affect development gene expression (FIGS. 11A and 1B). High resolution temporal maps of the transcriptional and chromatin environment in in vivo motor neurons during maturation reveal that the splicing factor, Mbnl2 is activated during postnatal motor neuron maturation (FIG. 2B). Together, these observations implicate Mbnl2 as a regulator of the adult splicing program and may contribute to determination of neuronal maturation stages. As shown in FIGS. 11H and 11I, Mbnl2-neurons display accelerated electrophysiological maturation. Mbnl2 expression in stem cell-derived neurons overcomes traditional challenges inducing 4R tau in patient lines (FIGS. 11J and 11K).

In one aspect, the platform technology involves overexpressing Mbnl2, a master regulator of developmental AS programs in neuron cells to produce mature neurons from differentiated stem cells. Accordingly, a method of generating mature neurons as well as a method of accelerating maturation of a neuron are disclosed. In some aspects, the methods are directed to generating mature motor neurons and accelerating maturation of motor neurons. In certain implementations, the neurons are differentiated from pluripotent stem cells.

In one implementation, the method of generating mature neurons produces mature neurons from differentiated stem cells. The method comprises differentiating pluripotent stem cells into neurons; and overexpressing Mbnl2 in the differentiated pluripotent stem cells. In some embodiments, Mbnl2 is overexpressed in the differentiated pluripotent stem cells by nucleofecting the differentiating pluripotent stem cell with a lentivirus construct for overexpressing Mbnl2. In some implementations, the method further comprises overexpressing Rbfox1 and/or reducing expression of PTBP1/2 in the pluripotent stem cells. In particular implementations, the method comprises simultaneously overexpressing Mbnl2 and Rbfox1 while reducing expression of PTBP1/2. In some aspects, the pluripotent stem cell expresses Cre. In some implementations, at least one lentivirus construct is used to overexpress the desired genes. In certain implementations, the lentivirus construct for overexpressing Mbnl2 has a sequence set forth in SEQ ID NO. 2 or SEQ ID NO. 3.

In another implementation, the method of generating mature neurons from pluripotent stem cells comprises conditionally expressing Mbnl2 in the pluripotent stem cells to produce transfected stem cells and differentiating the transfected stem cells into neurons. During the step of differentiating the transfected stem cells, expression of Mbnl2 in the cells are induced. In some embodiments, Mbnl2 is conditionally expressed in the pluripotent stem cells by transfecting the pluripotent stem cell with a conditional expression construct for overexpressing Mbnl2. In some implementations, the method further comprises overexpressing Rbfox1 and/or reducing expression of PTBP1/2 in the pluripotent stem cells. In particular implementations, the method comprises simultaneously overexpressing Mbnl2 and Rbfox1 while reducing expression of PTBP1/2. In some aspects, the pluripotent stem cell expresses Cre. In some implementations, at least one lentivirus construct is used to overexpress the desired genes. In certain implementations, the lentivirus construct for overexpressing Mbnl2 has a sequence set forth in SEQ ID NO. 2 or SEQ ID NO. 3.

In particular implementations, the method of accelerating maturation of a neuron, the method comprises simultaneously overexpressing Mbnl2 in the neuron; overexpressing Rbfox1 in the neuron; and reducing expression of PTBP1/2 in the neuron. In some embodiments, Mbnl2 is overexpressed in the neuron by nucleofecting the neuron with a lentivirus construct for overexpressing Mbnl2. In other embodiments, Mbnl2 is overexpressed in the pluripotent stem cells by transfecting the pluripotent stem cell with a conditional expression construct for overexpressing Mbnl2. In some aspects, the pluripotent stem cell expresses Cre. In some implementations, at least one lentivirus construct is used to overexpress the desired genes. In certain implementations, the lentivirus construct for overexpressing Mbnl2 has a sequence set forth in SEQ ID NO. 2 or SEQ ID NO. 3.

Also disclosed herein are constructs for inducing maturation of a neuron. In one aspect, the construct overexpresses Mbnl2. In another aspect, the construct overexpresses Mbnl2 and Rbfox1. In yet another aspects, the construct reduces expression of PTBP1/2 by knocking down expression of PTBP1/2 through reducing the amount of Ptbp1/2 mRNA transcript produced by the neuron. In certain implementations, the construct for inducing maturation of a neuron has as sequence set forth in SEQ ID NO. 2 or SEQ ID NO. 3.

A composition for inducing maturation of a neuron is further described. In some embodiments, the composition comprises the construct for overexpressing Mbnl2 and the construct for reducing expression of PTBP1/2. In other embodiments, the composition comprises the construct for overexpressing Mbnl2 and Rbfox1 and the construct for reducing expression of PTBP1/2. In some aspects, the construct for overexpressing Mbnl2 is a lentivirus construct, for example having the sequence set forth in SEQ ID NO. 2 or SEQ ID NO. 3. The composition is provided to a culture of neuron cells to induce maturation of the neuron cells. Accordingly, in some aspects, a kit for inducing maturation of a neuron is described. The kit comprises neuron cells that express Cre and the composition for inducing maturation of a neuron comprising a construct for overexpressing having the sequence set forth in SEQ ID NO. 2 or SEQ ID NO. 3.

As shown in FIGS. 5A-5D, MBNL proteins, especially Mbnl2, drives developmental splicing switch during neuronal maturation, in particular of MAPT exon 10 in both humans and mice. The timing of developmental increase in MAPT exon 10 splicing precisely coincides with the increase of Mbnl2 expression during mouse and human brain development (FIGS. 5C and 5D, left panels). Genetic depletion of Mbnl1/2 in the mouse brain (Mbnl1/2 double KO) or functional sequestration of Mbnl proteins in the postmortem brain of human patients with myotonic dystrophy (DM) dramatically reduces exon 10 inclusion (FIGS. 5C and 5D, right panels). MAPT exon 10 encodes the second of four microtubule-binding repeats and its alternative splicing generates tau isoforms containing three (3R) or four repeats (4R), which differ in microtubule binding affinity. Embryonic human brains express only 3R tau, while adult brains maintain a balance of 3R and 4R tau isoforms at about an equal molar ratio.

Tau aggregation forms neurofibrillary tangles (NFTs), a hallmark of multiple neurodegenerative diseases, such as Alzheimer's disease, and FTLD. Importantly, mutations within or around this exon that cause near complete exon 10 inclusion are sufficient to cause a familial form of FTLD. Thus, overexpression of Mbnl2 in neurons can be a model of studying tau pathology. Accordingly, a method of generating a tau pathology model is disclosed. This is a drastic improvement over existing technologies that model tauopathies. There are two primary obstacles that impede effective tauopathy modeling. The first is that human and rodent MAPT have divergent splicing patterns in multiple exons, including exon 10, which is constitutively included in the adult mouse brain, suggesting divergent tau function that makes animal models questionable. The second is that human iPSC-derived neurons predominantly express 3R tau, even after extended culture of one year. However, because overexpressing Mbnl2 leads to accelerated maturation of neurons and increased splicing of MAPT exon 10, the disclosed method of generating a tau pathology model using cell culture addresses the obstacle present in in human iPSC-derived neurons to model tauopathies.

In some implementations, the method of generating a tau pathology model comprise overexpressing Mbnl2 in the neuron. In other implementations, the method further comprises overexpressing Rbfox1 in the neuron and reducing expression of PTBP1/2 in the neuron. In particular implementations, the method comprises simultaneously overexpressing Mbnl2 and Rbfox1 while reducing expression of PTBP1/2.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1. Facilitating and Accelerating Spinal Motor Neuron Maturation with Mbnl2 Overexpression

The gene expression landscape of neurons has a program of highly regulated developmental alternative splicing (AS) switches. Thus, activating the adult splicing program in immature neurons should be sufficient to reprogram them to a mature state. Mbnl2 is a master regulator of the adult neuronal splicing program, and neurons with Mbnl2 loss-of-function display immature or embryonic properties. Therefore, overexpression of Mbnl2 is assessed for whether it is sufficient to alter neuronal maturity. First, mouse embryonic stem cells (mESCs) are differentiated into immature motor neurons (MNs). Mbnl2 expression in these cells and the maturation of these cells is assessed by evaluating expression of adult MN markers, cellular morphology and gene expression. Computational tools are also utilized to identify Mbnl2 AS gene targets that are involved in the acquisition of the mature MN fate.

Mouse motor neurons are differentiated and purified, overexpression of Mbnl2 in in vitro cultures sustained and cells collected for genomic analysis of splicing and gene expression patterns. To functionally evaluate whether Mbnl2-overexpressing motor neurons have accelerated maturation, developmentally specified cellular markers and features such as neurite length and complexity are examined.

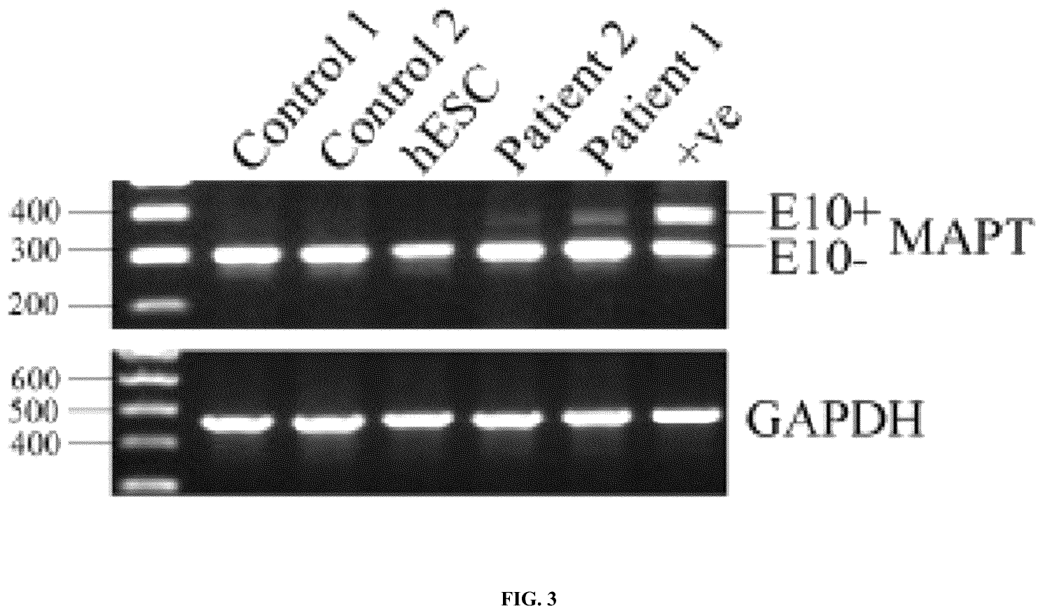

Examination of MNs in vivo indicates Mbnl2 expression increases postnatally and may drive the mature MN splicing pattern by directly binding its target transcripts (FIGS. 2B, 3B, 4A, and 4B). As MNs generated in vitro do not express Mbnl2 sufficiently during or after differentiation (FIG. 2B), driving early activation advances and accelerates adult splicing patterns and functionally matures MNs.

Mbnl proteins have recently been identified as a master splicing regulator that drives the developmental splicing switch during neuronal maturation, especially Mbnl2 (FIGS. 5A and 5B). In particular, Mbnl2 drives developing splicing of MAPT exon 10 in humans and mice (FIGS. 5C and 5D). Mbnl proteins bind to the downstream intron to activate exon 10 inclusion (FIG. 5B). The timing of developmental increase in MAPT exon 10 splicing precisely coincides with the increase of Mbnl2 expression during mouse and human brain development (FIGS. 5C and 5D, left panels). Genetic depletion of Mbnl1/2 in the mouse brain (Mbnl1/2 double KO) or functional sequestration of Mbnl proteins in the postmortem brain of human patients with myotonic dystrophy (DM) dramatically reduces exon 10 inclusion (FIGS. 5C and 5D, right panels). Mbnl-dependent regulation of MAPT exon 10 splicing is validated using minigene systems by co-expression or knockdown of Mbnl expression in HEK293T cells (FIG. 5E).

The impact of Mbnl proteins on maturation-dependent splicing switches is genome-wide. Among exons showing conserved, late splicing switches during mouse and human brain development, 65% and 45% are regulated by Mbnl2 to activate their adult splicing patterns (FIG. 5A). Accordingly, Mbnl2 overexpression in nascent neurons is expected to facilitate their maturation by driving the adult splicing program.

A. Establishing a Mouse Embryonic Stem Cell Line with Doxycycline-Inducible Mbnl2 and Motor Neuron Reporter

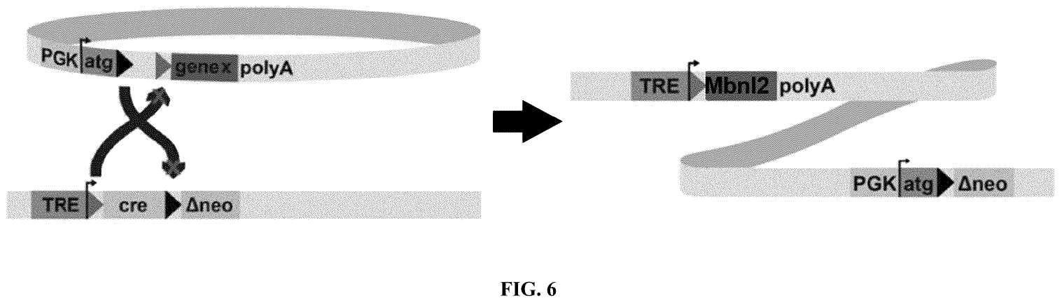

As downstream experiments require MNs, a system that is compatible with MN purification was used. A previously established an mESC line contains a MN reporter (Hb9::GFP, for sorting MNs) and an Inducible Cassette Exchange (ICE) locus (FIG. 6). Upon cassette exchange, the Cre is replaced with a gene of interest and Aneo is repaired, allowing selection of mESCs with the desired recombination event. A FLAG-tagged Mbnl2 template is cloned, nucleofected into mESCs, induced exchange and cells with correct Mbnl2 insertion selected. Sanger sequencing is used for confirmation. 4-5 iMbnl2 clones are characterized.

### B. Engineering Lentiviral Constructs for Rbfox1 Overexpression and Ptbp Knockdown

Another way to express Mbnl2 in mouse ESC-derived motor neurons utilizes a lentivirus delivery system. Mbnl2 expression has been shown to suppress lentivirus packaging when the Mbnl2 ORF is on the sense strand of the viral RNA, but this likely because overexpressed Mbnl2 bind its own transcript. To overcome this technical limitation, a FLEx (or Flip excision) Mbnl2 and FLEx-GFP (control) lentivirus constructs was built that express BFP in the default setting, but in the presence of Cre recombinase, it activates FLAG-tagged Mbnl2 or GFP expression and turns BFP off (FIG. 11C). The sequence of the FLEX-GFP lentivirus sequence is reflected in SEQ ID NO. 1. The sequences of FLEx Mbnl2 lentivirus constructs are reflected in SEQ ID NOs. 2 and 3. Selective and robust Mbnl2 overexpression in HEK293T cells transfected with FLEx-Mbnl2 and Cre recombinase was confirmed by immunoblots (FIG. 11D).

To overexpress Mbnl2 using the FLEx virus in motor neurons, a source of Cre is needed. To this end, a mouse ESC line was previously derived (Chat-Cre), which has a Cre knocked into the choline acetyltransferase (Chat) locus and thus specifically expresses Cre in motor neurons and other cholinergic cells. These Chat-Cre ESCs have been differentiated into motor neurons in embryoid bodies (EBs) using well-established protocols and infected cultures with FLEx-Mbnl2 or controls upon dissociation into a single cell suspension (FIG. 11D). Serial dilutions of virus were conducted to identify conditions where most motor neurons expressed Mbnl2 or control GFP without affecting cell viability or causing transgene aggregation. At these optimized conditions, post-mitotic motor neurons expressing Mbnl2 were cultured for additional 2, 7 or 14 days (DIV2, DIV7, and DIV14) to develop an understanding of Mbnl2-dependent splicing changes.

Per condition, 1M motor neurons per well were cultured in 6-well dishes for RNA analyses and ˜60K motor neurons on 12 mm glass coverslips in 24-well plates for immunostaining. Immunostaining of cultured motor neurons confirmed Mbnl2 expression and its expected nuclear localization (FIG. 11F). Moreover, splicing of Mbnl2 target cassette exons previously identified was examined, including exons in Mapt, Ank3 and Kcnma1 genes. As shown in FIG. 11G, ectopic Mbnl2 expression starts to activate their mature splicing patterns by DIV7 and further potentiates it by DIV14.

These experiments will be repeated in 6-well (1M motor neurons, protein), 12-well (500K motor neurons, RNA) and 24-well (60K motor neurons on 12 mm glass coverslips, immunostaining) formats to quantify ectopic Mbnl2 expression in the above-described stem cell-derived motor neurons at different time points by RT-qPCR, immunostaining and immunoblots.

In parallel splicing maturation across in vitro timepoints and conditions are evaluated through RT-PCR of an expanded set of Mbnl2-regulated cassette exons, a subset of which are in FIG. 11G. Exon inclusion from in vivo adult motor neurons from data previously collected are used as maturation landmarks. FIGS. 11A and 11B display the comparison between Mbnl2-expressing stem cell-derived motor neurons and adult motor neurons.

### C. Interrogating Sustained Mbnl2 Expression in Motor Neuron Cultures

Conditional Mbnl2 expression is critical as Mbnl2 decreases pluripotency in stem cells. Furthermore, previous work has found that common promoters are usually silenced in postmitotic neurons. Therefore, it is examined first if the Tet-responsive promoter is active for the duration of the tests. MNs are generated from 5 iMbnl2 lines using previously developed methods and Mbnl2 is induced early in progenitor MNs. Mbnl2 expression is examined through immunostaining. Early interrogation of Mbnl2 allow selections of ideal clones for further assessment.

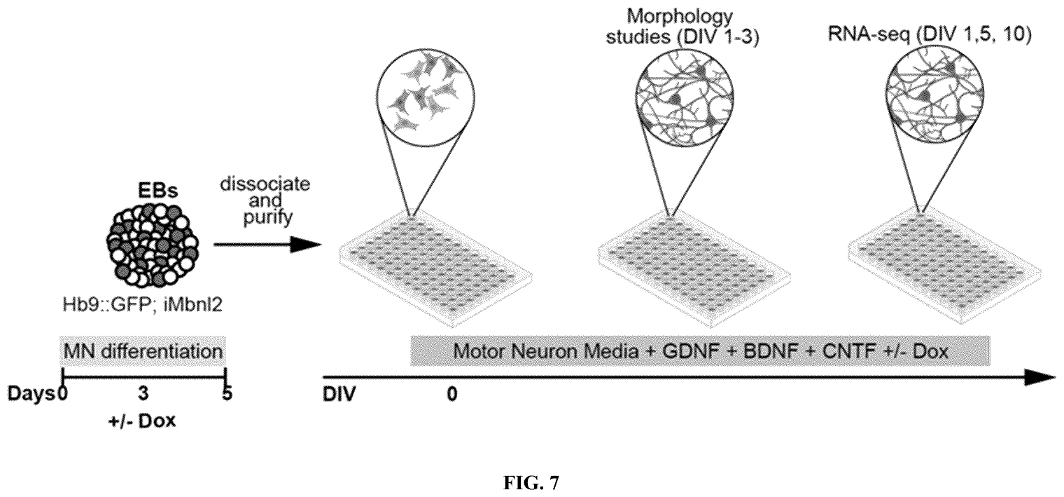

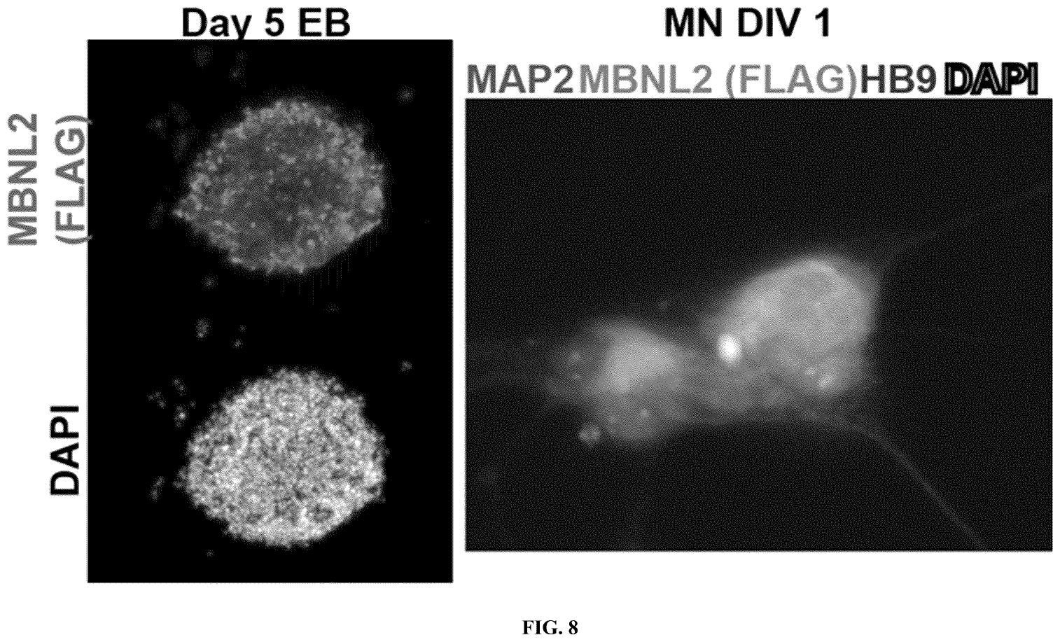

Maturation study design is shown in FIG. 7. Mbnl2 expression is induced on late day 3/early day 4 of the differentiation protocol, MNs collected on day 5 by dissociating embryoid bodies (EBs) and sorting GFP+ cells with FACS, and culture MNs on 96-well glass bottom plates coated with poly-L-ornithine and Laminin. 1000-2000 MNs/well are cultured in motor neuron medium supplemented with doxycycline and neurotropic factors. It has been found previously that a higher MN density causes cell clumping and presents difficulties for identifying individual cells. The same strategy without doxycycline (Dox−) will serve as negative control. Mbnl2 expression and (leakiness) are evaluated at 1, 5, and 10 days in vitro (DIV) through immunostaining and microscopy.

D. Transcriptomic Analysis, Splicing Quantification and Integration with In Vivo MN Maturation

RNA from MNs cultured for 1, 5 and 10 DIV in 6-well dishes is collected and gene expression and splicing patterns mapped by cloning stranded, ribo-zero plus, total RNA sequencing libraries (RNA-seq). All sequencing experiments are performed in biological triplicates, derived from independent differentiation experiments. Dox-culture conditions is used as negative control. In-depth analysis of RNA-seq data is performed using multiple tools, including DESeq2 for gene expression, and MAJIQ and Quantas for splicing. A recent report identified genes that are activated and downregulated in adult motor neurons during maturation. This resource is used to examine if Mbnl2 regulates in vivo markers of motor neuron development. These markers are also validated using a complementary immunostaining approach. Similarly, it has been previously cataloged splicing dynamics during motor neuron maturation and software developed to stage neuronal maturation using splicing profiles (FIG. 4A, Splicescope). A panel of RNA-seq datasets is integrated from in vivo and in vitro neurons (w/wo genetic manipulations), along with the datasets proposed above to predict the maturation stage of Mbnl2-overexpressing MNs and place within the context of previously studied manipulations.

### 1. Bulk RNA-Seq Analysis.

To obtain a comprehensive view of splicing and gene expression changes in motor neurons with Mbnl2 overexpression, bulk RNA-seq profiles from FLEx-Mbnl2 and control treated Chat-Cre motor neurons will be generated at DIV2, DIV7 and DIV14 cultures in triplicates (˜60 million 2×150 nt paired end reads per sample). RNA-seq data will be analyzed using Quantas, an established bioinformatics pipeline developed to identify gene expression and splicing changes upon Mbnl overexpression as well as between the different timepoints. This dataset will be compared with in vivo motor neuron developmental time course, which was previously generated. Principal component analysis (PCA) will be employed to assess global trends in splicing and gene expression profiles. Splicescope will be used to evaluate whether global splicing and gene expression patterns in Mbnl2-expressing neurons reflect an overall shift towards the mature transcriptome profile, and to what extent, as has been done previously (FIGS. 4A-4B).

2. SnRNA-Seq and snATAC-Seq Analysis.

Although only Chat-cre+ motor neurons express viral Mbnl2, bulk analysis includes a minor fraction of other cells that are spinal interneurons or glial cells. Furthermore, motor neurons acquire different molecular subtype identities that differ in their connectivity and function. To evaluate with finer granularity the ability of Mbnl2-dependent splicing programs to activate the mature transcriptomic and epigenetic state in cultured motor neurons, the bulk RNA-seq analysis will be complemented with single nuclei RNA-seq and ATAC-seq analysis, as performed in previous studies. Briefly, this analysis will be performed on 6000-8000 nuclei per run in biological triplicates at DIV14 when greatest changes in splicing are expected. The isolated nuclei will be visually evaluated using fluorescence microscopy after DAPI staining to check morphology, ensure that DNA has not leaked out of nuclei, and assess formation of clumps. snRNA- and ATAC-seq runs will be performed by the Columbia Genome Center using the 10× genomics Multiome pipeline. Cellranger software (part of the 10× pipeline) will be used to align the sequenced reads and quantify gene expression and chromatin accessibility peaks. Seurat and Signac will then be used to cluster the cells, identify cells that express viral Mbnl2, and perform differential gene expression and chromatin accessibility analysis. The maturation-dependent genes previously identified during in vivo motor neuron maturation will be examined to determine which motor neuron subtypes exhibit a more mature expression profile. Finally, differential chromatin accessibility data will be leveraged to identify sets of putative regulatory regions and enriched transcription factor motifs driving the gene expression changes downstream of Mbnl2 expression.

3. iCLIP Analysis.

In parallel, individual-nucleotide crosslinking and immunoprecipitation (iCLIP) analysis will be performed to map Mbnl2 binding sites using antibodies against the FLAG tag or the Mbnl2 protein using cells from DIV14 culture in triplicate (˜30 million single-end reads per sample). CLIP data analysis will be performed using the established software CLIP Tool Kit (CTK) previously developed. It will be confirmed Mbnl-binding underlies the splicing switching of its target exons, as well as compare whether there is any RNA-binding difference between endogenous and ectopically expressed Mbnl2.

Jointly, these experiments will address whether Mbnl2 overexpression is sufficient for 1) splicing switching of its target exons through direct binding and regulation; 2) splicing switching of developmentally regulated exons that are not direct Mbnl2 targets; and 3) switching of transcriptomic and epigenetic profiles not directly affected at the splicing level.

### E. Functional Readouts of Neuronal Maturity

### 1. Morphometric Analysis.

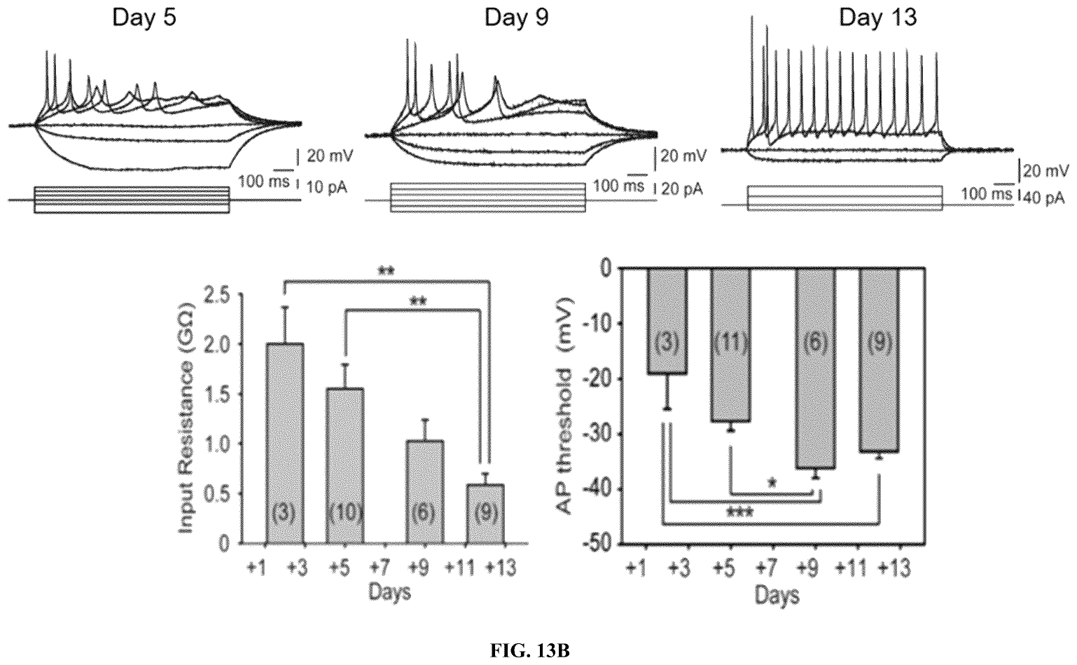

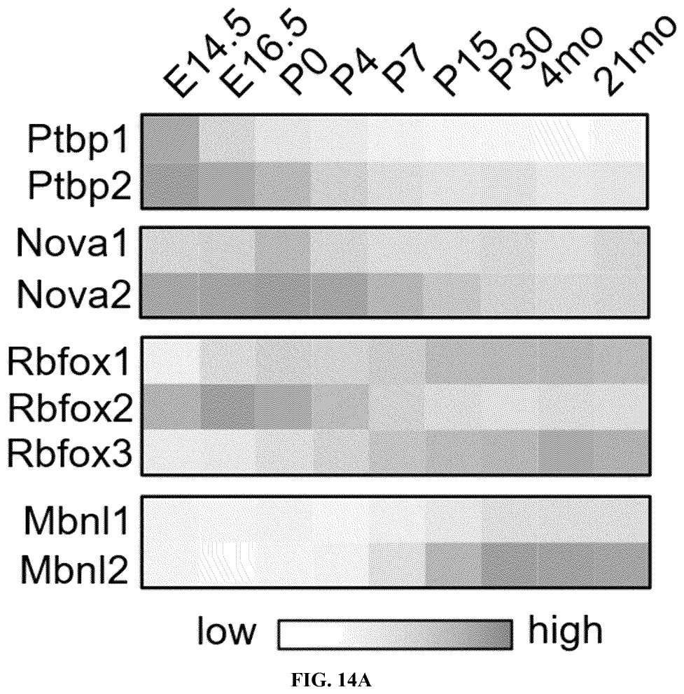

To investigate whether Mbnl2 over expression promotes morphological maturation of motor neurons, detailed longitudinal morphometric analysis of dissociated motor neuron cultures will be performed. Control and Mbnl2 transduced motor neurons will be plated at a low density (50-100 cells per well) on 96-well Greiner plates coated with polyethyleneimine and Matrigel®. Plates will be counterstained with Calcein-AM dye (Invitrogen™) and imaged on DIV2, DIV7, and DIV14, followed by fixation and immunostaining for ISL1/MNX1 (pan motor neuron markers). Images will be analyzed using Metamorph® software (Molecular Devices) to provide automatic and unbiased quantification of motor neurons on several metrics, such as cell soma size, total neurite length, number of processes and branches, as performed previously (FIG. 13A).

### 2. Electrophysiological Characterization.

To evaluate effects of Mbnl2 overexpression on electrophysiological properties of motor neurons, differentiated WT and Mbnl2 transduced motor neurons will be plated at high density (15,000 motor neurons per well) onto a 96 well multielectrode array (MEA) plate. Cultures will be recorded three times a week for a period of 3 weeks to measure the onset and progression of spontaneous electrophysiological activity. In the second set of experiments, motor neurons will be plated on glass coverslips and cultured for 2, 7 and 14 days at which points individual motor neurons will be evaluated by current clamp. Passive membrane properties, repetitive firing patterns, action potential characteristics, and excitability will be measured in 15 motor neurons per cell line, as done previously (FIG. 13B). In addition, synaptic activity will be examined using whole-cell voltage clamp and probe the types of synaptic inputs using blockers of cholinergic, glutamatergic and GABAergic receptors.

### Example 2. Altering MAPT/Tau 3R: 4R Ratio in Neurons to Better Model Tauopathies by Regulating Mbnl2 Activity

It has been shown that Mbnl2 developmentally induces adult tau isoforms through AS. So Mbnl2 induction can be used to accelerate neuron maturation and produce adult tau, which results in a new in vitro model to study tau.

Cortical neurons from human induced pluripotent stem cells will be generated (iPSCs), infect these with an inducible Mbnl2 virus and drive increase of MAPT exon 10 by stimulating Mbnl2 expression. Targeted elements that regulate exon 10 inclusion within the endogenous MAPT gene will be screened and antisense oligonucleotides (ASOs) that decrease exon 10 inclusion and 4R tau levels identified. Finally, it will be assessed if ASOs can decrease 4R tau and tau aggregation in patient-derived neurons with naturally elevated 4R tau.

### A. Construction and Validation of an Inducible Mbnl2 Lentivirus

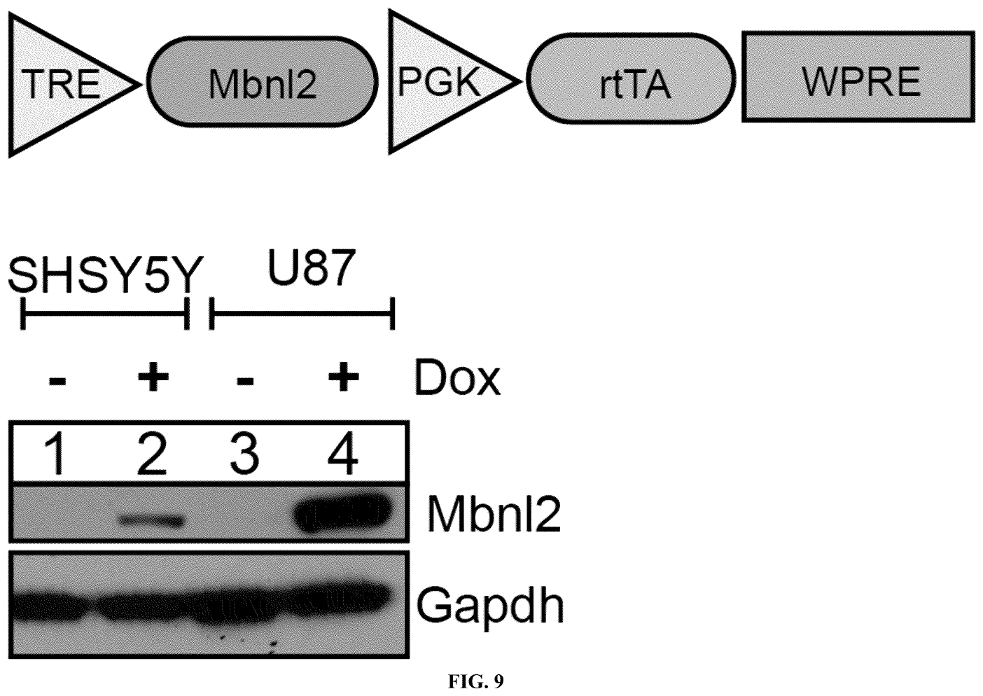

A FLAG-tagged Mbnl2 is cloned into a doxycycline-inducible lentivirus backbone that contains a constitutive transactivator (rtTA) (FIG. 9). The virus is validated by infecting SHSY5Y and U87 cells and testing Mbnl2 expression with and without doxycycline induction. As these cells express MAPT, the activity of Mbnl2 is evaluated by assessing MAPT exon 10 splicing by rt-PCR.

B. Increasing 4R Tau Expression in Excitatory Neurons with Mbnl2 Overexpression

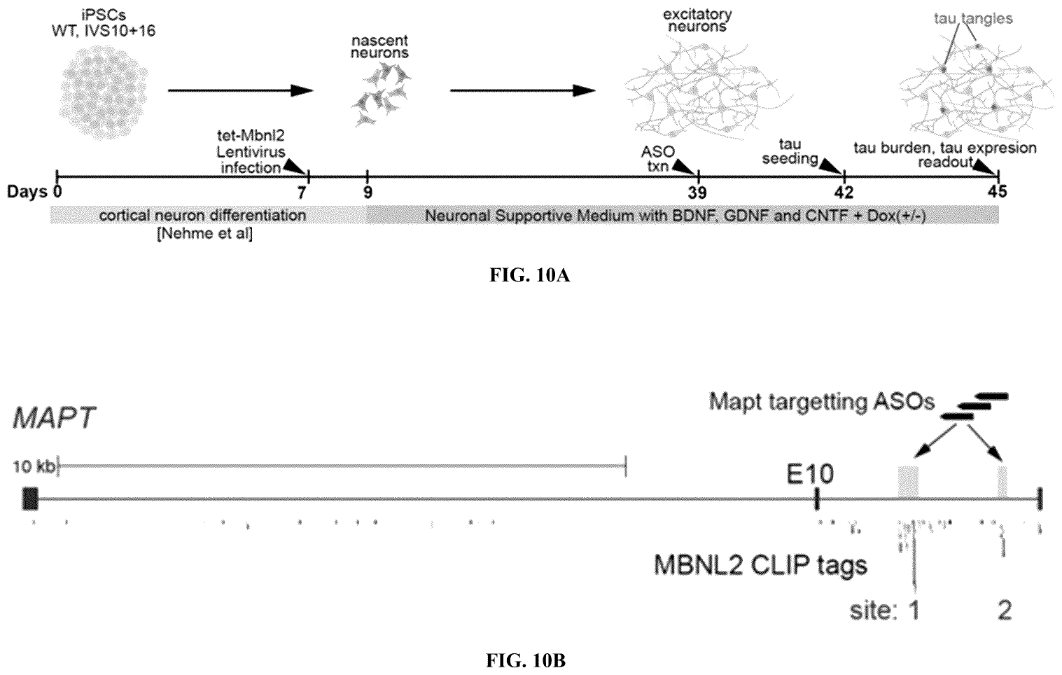

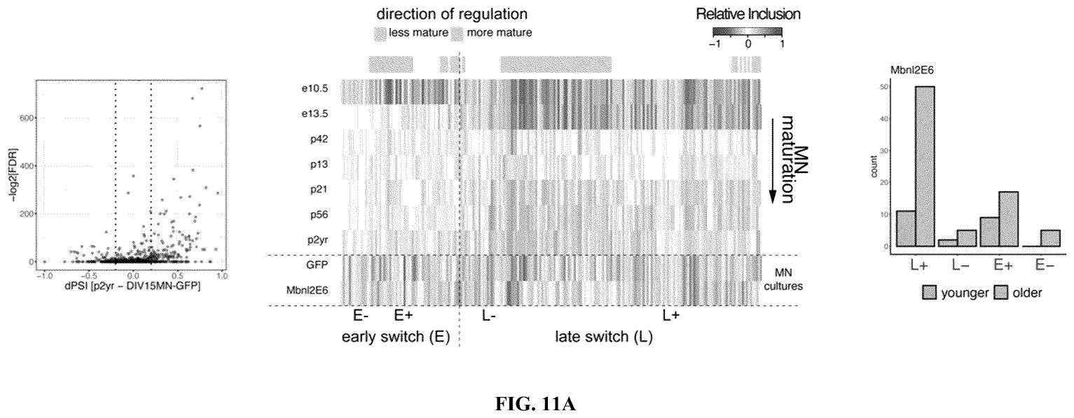

A previously developed protocol had been used in the past to produce cortical-like, excitatory neurons by treating iPSCs to a combination of directed differentiation and transcription-factor programming. Cortical neurons will be generated using this protocol and infect postmitotic neurons with Mbnl2 lentivirus (FIG. 10A). Mbnl2 expression will be confirmed by immunostaining. MAPT exon 10 inclusion will be assessed by rt-PCR and 3R/4R-tau expression by western blot. These experiments as well as those below will be expressed in biological triplicates. Relevant bands will be quantified to allow statistical comparison.

C. Screening Candidate Splice Modulating ASOs that Alter Tau 3R: 4R Ratio

Instead of screening the kilobases of intron that surround exon 10, two SREs previously identified will be targeted (FIG. 10B). The combined length of these elements is approximately 300 nt, which will be tiled by 15-20 complementary ASOs (FIG. 10B). ASOs will be delivered into Mbnl2-overexpressing cortical neurons plated in 12-well dishes via Lipofectamine™ RNAiMAX (ThermoFisher). The ASOs will be 20 nt long and 2′-O-methoxy-ethyl modified to enhance target engagement and stability in cells. MAPT transcript and tau protein will be assessed as described above.

### D. Using ASOs to Decrease Reduce 4R Tau in MAPT IVS10+16 Neurons

Familial forms FTLD can be directly linked to mutations in the MAPT gene. Several of these mutations, such as IVS10+16 have no effect on protein sequence but increase exon 10 inclusion and hence 4R tau, in the brain as well as neurons generated in vitro. iPSCs with the MAPT IVS10+16 mutation were obtained from the Tau Consortium Stem Cell Group. Cortical neurons will be generated from these cells, with Mbnl2 overexpression and confirmed elevated 4R tau. Successful ASOs will be transfected from the screen above to evaluate their ability to decrease inclusion of exon 10. Scrambled ASOs will serve as negative controls (FIG. 10A).

### E. Confirming a Role of Mbnl2 and ASOs in Modulating Tau Pathology in Human Neurons

4R tau is more susceptible to aggregation in vivo and in vitro. Human stem cell-based neuronal models do not express 4R tau except for cell lines with select MAPT mutations, impeding a thorough study of human 4R tau biology. To evaluate if Mbnl2-overexpression neurons can be utilized as cells of interest for this purpose, cell-based tau-seeding assays will be conducted which evaluate tau aggregation potential. Wildtype iPSC-derived cortical neurons will be cultured with and without doxycycline and include tau seed in the culture medium (FIG. 10A). Tau aggregation will be measured by immunostaining with tau antibodies and counting cells with tau tangles. To ensure unbiased quantification the evaluation will be under blind experimental conditions. It will also be assessed if use of 4R reducing ASOs in Mbnl2-overexpressing and/or MAPT IVS10+16 neurons will decrease tau aggregation. These experiments will be conducted as described above and shown in FIG. 10A. For each cell line, a scrambled ASO will serve as negative control.

### Example 3. Accelerating In Vitro Mouse Motor Neuron Maturation Through Simultaneous Overexpression of Mbnl2, Rbfox1 and Knockdown of Ptbp1/2

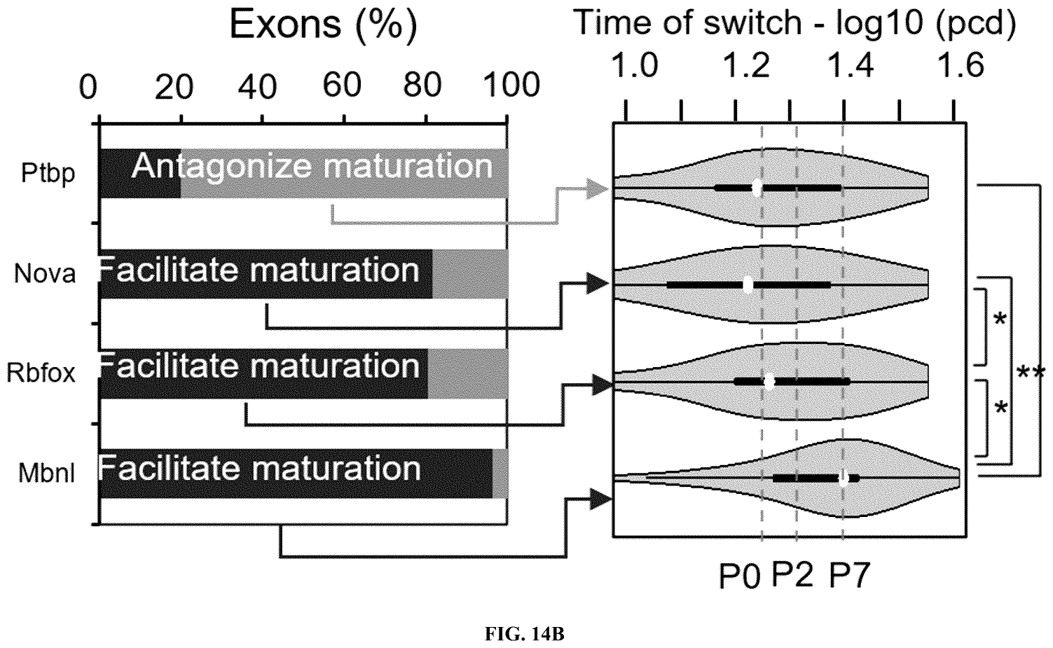

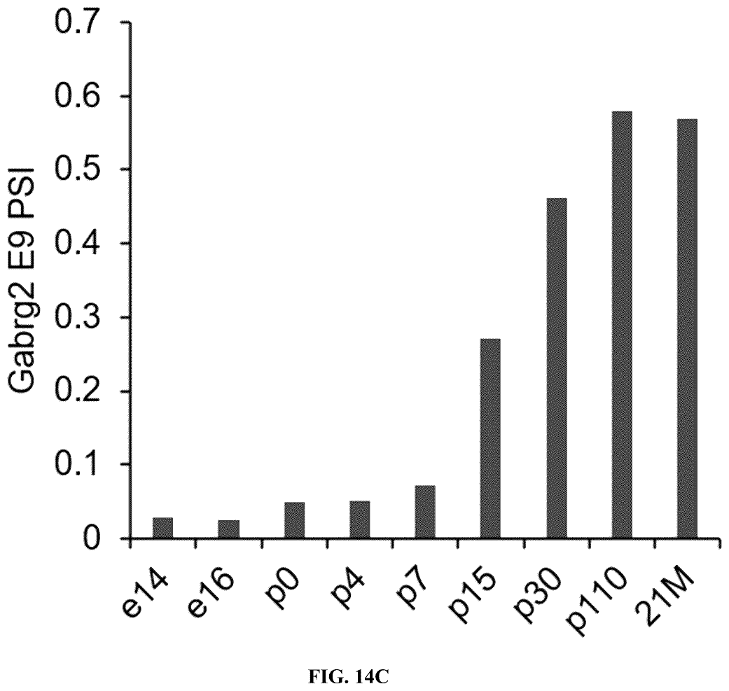

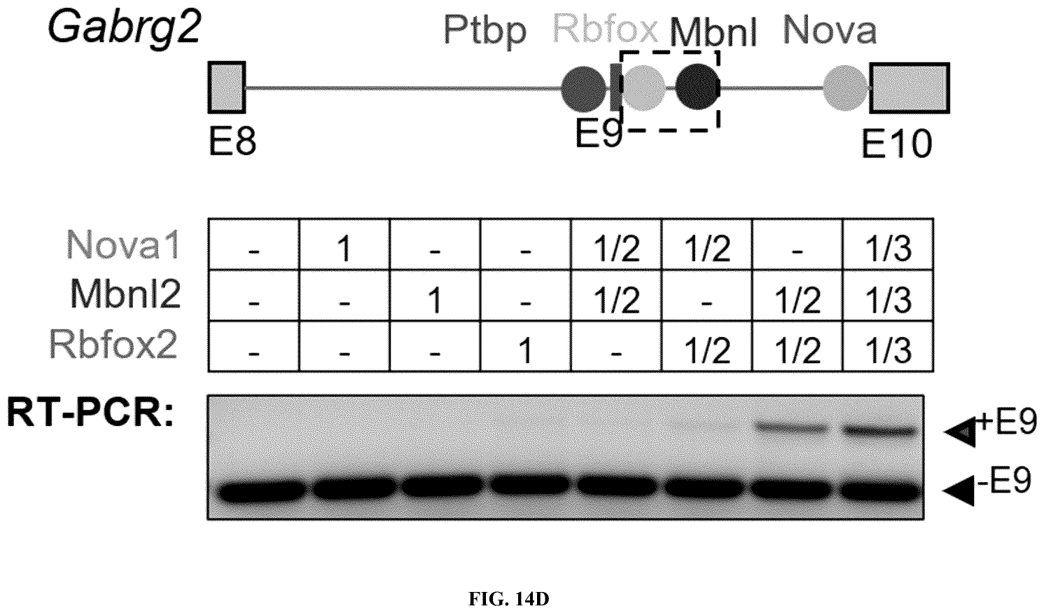

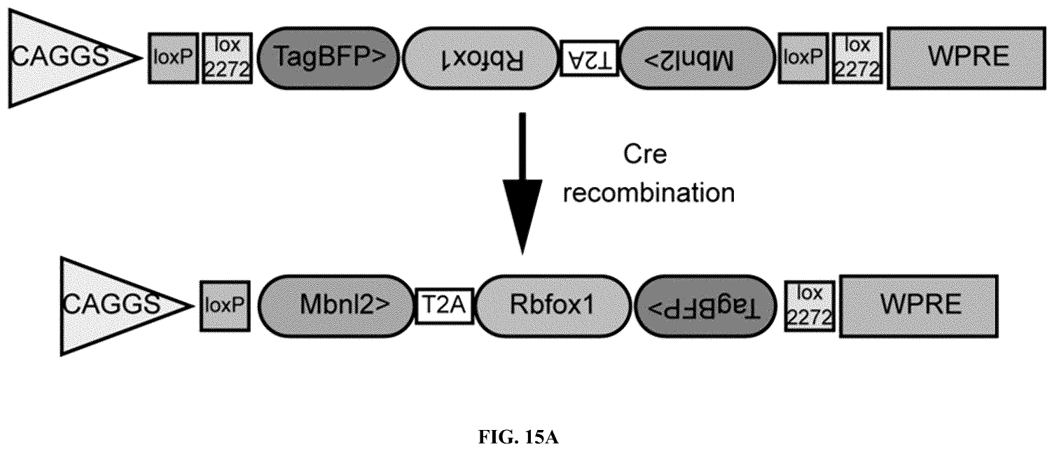

While Mbnl proteins regulate a significant proportion of developmentally regulated cassette exons, they may not be sufficient to activate the complete set of adult splicing isoforms required for neuronal maturation (FIGS. 14A-14D). Moreover, Mbnl activity might also be modulated by cofactors. Consistent with this notion, Mbnl2 overexpression is not sufficient to drive adult splicing switches in DIV2 motor neurons, suggesting that at this early time point, certain cofactors required for Mbnl2 activity are absent or repressors of Mbnl activity are present. Therefore, manipulation of multiple developmental splicing factors will drive more complete and robust splicing shifts towards the adult patterns, in analogous to Yamanaka factors in driving iPSC reprogramming. Thus, motor neuron maturation will be advanced by testing permutations of Mbnl2 and Rbfox1 co-overexpression with simultaneous Ptbp1/2 knockdown.

### A. Engineering Lentiviral Constructs for Rbfox1 Overexpression and Ptbp Knockdown

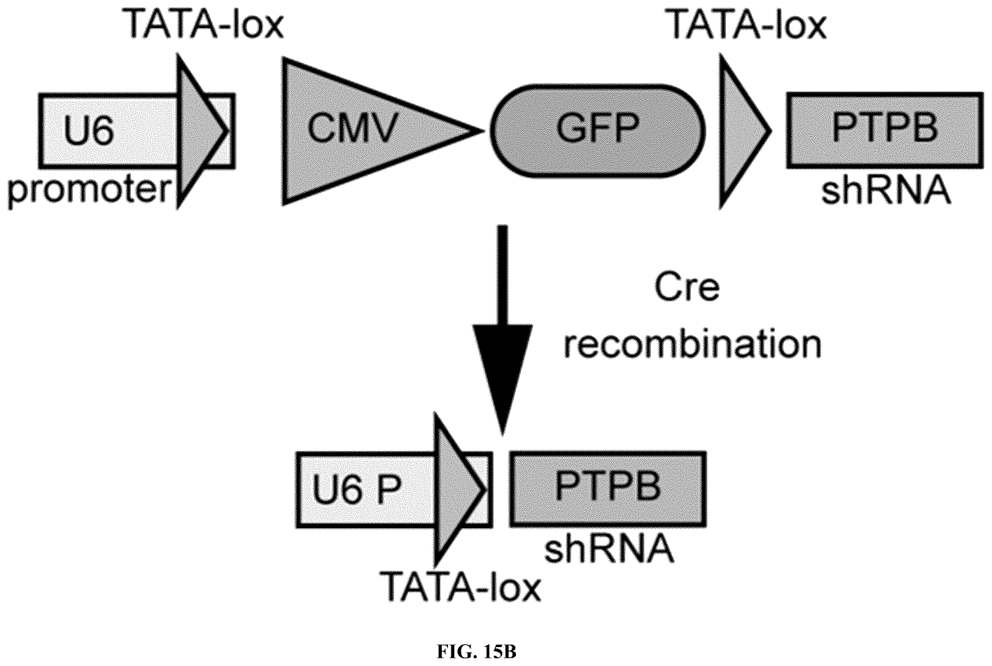

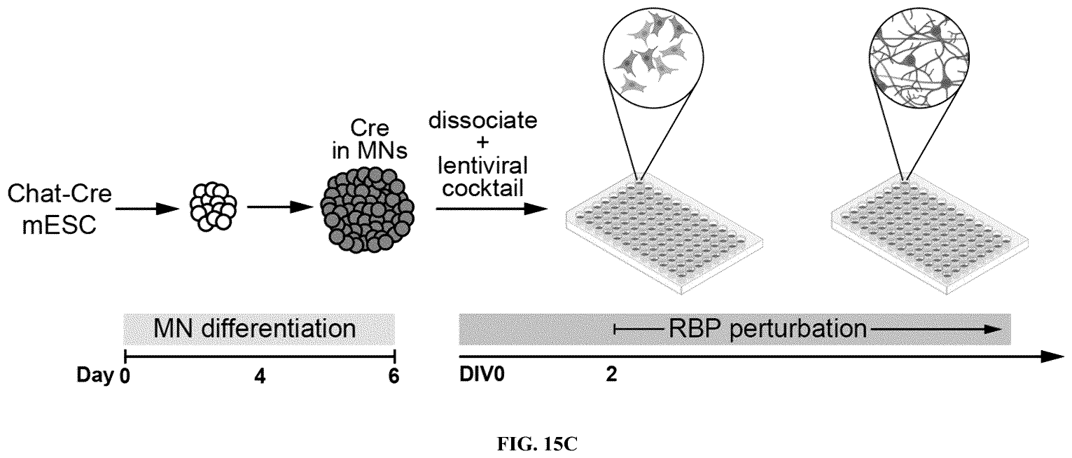

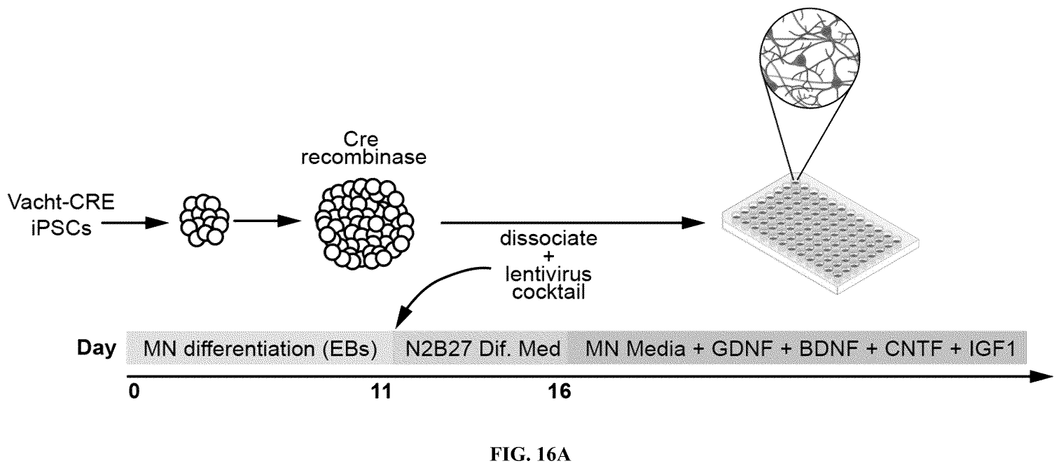

Evaluating combinations of RBPs for their ability to facilitate and accelerate motor neuron maturation will be aided by the development of new lentivirus constructs. The use of the FLEx lentiviral construct that is in the off-state in non-motor neuron cell types within the cultures will be continued; this construct has been extensively validated to transduce motor neurons efficiently. Since it has been proposed to overexpress Mbnl2 and Rbfox1, a FLEx-Rbfox1 construct that will individually express Rbfox1 will be built, as well as modify the FLEx-Mbnl2 construct into FLEX-Mbnl2-t2A-Rbfox1 allowing simultaneous expression of the two RBPs (FIG. 15A). Having the same source for Mbnl2 and Rbfox1 will ensure that all transduced cells co-express the desired proteins. Neuronal Rbfox1 cDNA has been previously cloned and verified that it is expressed in the nucleus in motor neurons.

This Rbfox1 cDNA will be subcloned into the FLEX-system. As the FLEx-Mbnl2 has a FLAG tag, an N-terminal HA-tag for Rbfox1 will be used to uniquely study exogenous Rbfox1. For knockdown of Ptbp1/2, the pSico lentiviral scaffold will be utilized, which conditionally expresses shRNAs under Cre expression (FIG. 15B). This shRNA backbone utilizes a ubiquitous U6 promoter driving shRNA expression and an interfering CMV-driven GFP transgene flanked by modified Cre substrates called TATA-lox sequences. In the presence of Cre, TATA-lox sequences recombine to excise CMV-GFP, reorienting the shRNA immediately downstream of the U6 promoter. In this setting, shRNAs are strongly transcribed by Pol III to enhance knockdown efficiency. Since it is being proposed to knockdown Ptbp1/2 in mouse and in human motor neurons, shRNAs that target Ptbp1/2 in regions conserved between mouse and human will be used. The constructs for ectopic Rbfox1 and Ptbp1/2 silencing will be validated in HEK293T (human) or Neuro-2A cells (mouse) by co-expressing with a Cre plasmid, as performed in previous experiments (FIG. 11B).

### B. Combinatorial Perturbation of Mbnl2, Rbfox1 and Ptbp1/2 in Motor Neurons

To evaluate combinatorial effects of the candidate RBPs on maturation-dependent splicing program, the Chat-Cre ESC line will be differentiated into spinal motor neurons and transduce the cells with Rbfox1, Mbnl2 and Ptbp1/2 lentiviruses. The same strategy used above will be used, in which Chat-Cre ESCs are differentiated into motor neurons, dissociated into single cell suspensions on day 5 and infected with lentiviruses at the time of their plating on laminin-coated plates (FIG. 15C). For the proposed studies, lentiviruses will be tested individually and in pooled configurations. Individual conditions consist of Mbnl2 or control GFP overexpression only, Rbfox1 overexpression only, Ptbp1 knockdown only or Ptbp2 knockdown only. Different permutations of pooled perturbations will be attempted, such as co-expression of Mbnl2 and Rbfox1, and overexpression of Mbnl2 (or Rbfox1) with downregulation of Ptbp1/2, and also test the complete pooled configuration, namely, co-expression of Mbnl2 and Rbfox1 and simultaneous knockdown of Ptbp1 and Ptbp2. Using a ratio of 1 motor neuron to 200 viral particles (as quantified by Lenti-X™ qRT-PCR Titration Kit) yields a near 100% transduction rate with minimal consequences on motor neuron viability.

˜60K motor neurons will be cultured on 12 mm glass coverslips to immunostain motor neurons for expression of Mbnl2, Rbfox1 and Ptbp1/2. In parallel, ˜1M motor neurons per condition will be cultured in 6-well plates to collect RNA for transcriptomic profiling. Cultures will be examined at the DIV2, DIV7 and DIV14, the same timepoints used for Mbnl2 overexpression, to allow comparison between different conditions.

Expression of endogenous and exogenous RBP expression will be monitored by RT-qPCR and immunoblots, as well as splicing switches of previously identified RBP targets.

Bulk RNA-seq will be conducted on these samples in biological triplicates to allow global examination of gene expression and alternative splicing. Gene expression and alternative splicing profiles will be mapped onto the in vivo neuron maturation trajectory, to identify RBP combinations that yield the most mature cells.

Once the optimal condition is identified, snRNA-seq and snATAC-seq will be performed at DIV14 to evaluate mature transcriptome and chromatin accessibility at the single cell level.

iCLIP of Rbfox1 and Mbnl2 will be also performed, as described previously. Ptbp1/2 iCLIP will be performed in control motor neurons at the same time points without knockdown. RBP binding sites mapped by iCLIP will be overlaid with genes with altered splicing and expression to distinguish direct vs. indirect regulation.

It will be evaluated if most effective RBP combinations can further accelerate the morphological and electrophysiological maturation of cultured motor neurons by live imaging, patch clamp and MEA analysis.

### Example 4. Accelerating In Vitro Maturation of Human iPSC-Derived Motor Neurons with RBPs

Manipulating the same set of key developmental splicing factors, namely simultaneous overexpression of MBNL2 and RBFOX1 and knockdown of PTBP1/2 in nascent neurons will also be sufficient to promote human neuron maturation. These RBPs show similar developmental expression changes, which are accompanied by paralleled splicing switches of the core sets of target exons, suggesting molecular programs underlying neuronal maturation are conserved in both systems (FIGS. 5A-5E). Human iPSC-derived spinal motor neurons will be modified to overexpress MBNL2 and RBFOX1 as well as knockdown PTBP1/2. It will also be tested whether mature motor neurons produced with this approach can be used to better model late-onset motor neuron diseases, using ALS/TDP43 pathology as a model system for a proof of principle.

C. Accelerating Human Motor Neuron Maturation with MBNL2, RBFOX1 and PTBP1/2 Modulation

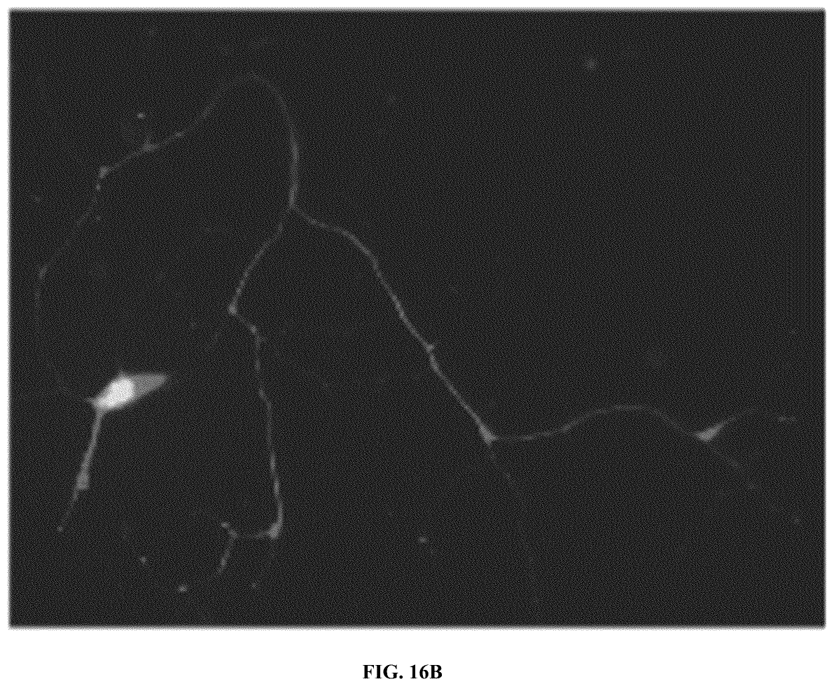

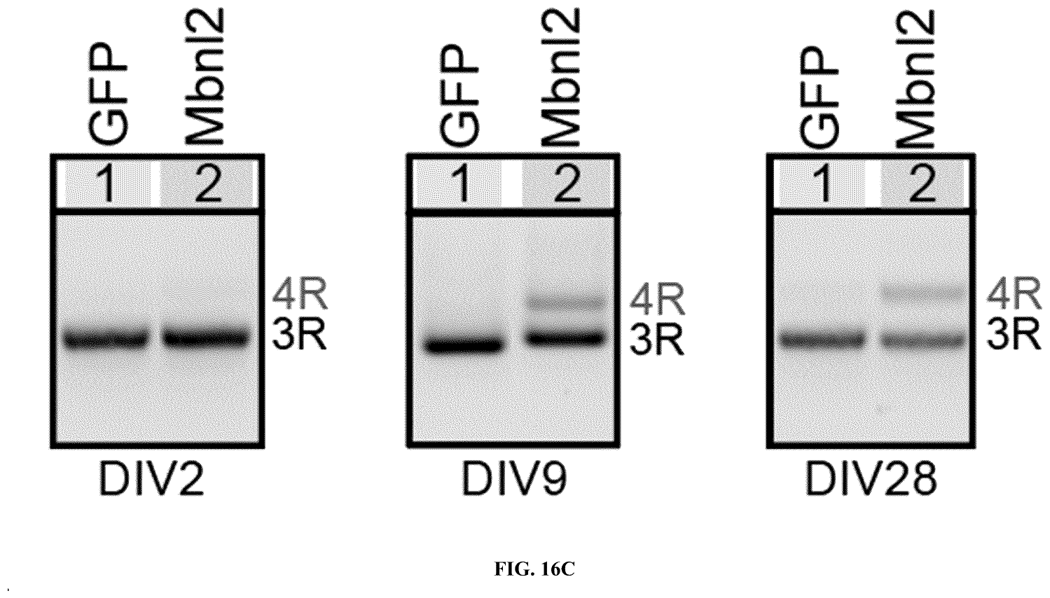

Like in studies of mouse neurons, the impact of RBP perturbation in human motor neurons will be investigated. The lentiviral FLEx-MBNL2, FLEx-RBFOX1 and FLEx-MBNL2-t2A-RBFOX1 will be reengineered using human cDNAs to test maturation in human cells (FIGS. 16A-16C). The Ptbp1/2 shRNAs (pSico-Ptbp1/2, FIG. 15B) will be recycled for human experiments as they target conserved sequences. The CAGGs and U6 promoters used in these constructs are known to be robustly active in human motor neurons. However, to utilize these expression vectors a cell line will be required which expresses Cre in motor neurons. It has been previously reported a human iPSC line (Vacht-Cre) harboring a Cre knock-in under control of the Vesicular Acetylcholine Transporter (VACHT) gene promoter, which expresses Cre in motor neurons and other cholinergic cell types. These Vacht-Cre iPSCs will be differentiated into motor neurons, and transduce dissociated, nascent motor neurons with MBNL2 and RBFOX1 overexpression and PTBP1/2 knockdown lentiviruses in individual and pooled iterations.

Maturation of human and mouse neurons occurs on different timescales, with human neurons requiring significantly longer time in vivo to achieve transcriptomic and functional maturity. Therefore, the temporal dynamics of alternative splicing in human motor neurons in vitro cultured for varying periods (DIV7, 14, 28, and 56) will initially be estimated by using RT-PCR to examine key target cassette exons of each RBP identified previously. The timepoint post-transduction when the maturation splicing patterns are induced will be delineated, how these events change with time thereafter, and when ceiling effects are observed. These timepoints will serve as landmarks for transcriptomic analyses, which will be systematically conducted by RNA-seq in triplicates. Global gene expression and splicing changes will be examined and map them onto well-defined in vivo neuronal maturation trajectories. An algorithm named Splicescope has been developed to predict neuronal maturation stages based on developmental gene expression and splicing profiles. The Splicescope algorithm was adapted to model human neuronal maturation, which will be used to determine whether the global splicing and gene expression pattern suggest a shift towards the mature identity. These analyses will be used to compare combinatorial effects of RBPs within in vitro conditions. Finally, snRNA-seq, snATAC-seq and iCLIP analysis will also be performed at the final time points to confirm the impact of RBP manipulation in different cell subtypes and distinguish direct vs. indirect regulation, similar to those proposed for mouse motor neuron.

Human motor neurons from Vacht-Cre iPSCs have been already generated, which were then infected with the FLEx-Mbnl2 virus generated and cultured further for up to 28 days (DIV28) (FIG. 16A). Nuclear Mbnl2 protein expression was detected (FIG. 16B) and increased MAPT exon 10 inclusion (i.e., increased 4R) as early as DIV 9 in culture (FIG. 16C). Importantly, using this strategy, more 4R tau was generated than previous studies in which stem cell-derived human neurons were cultured for a full year. This suggests overexpression of RBPs can overcome posttranscriptional barriers, which cannot be accomplished by simply aging neurons in vitro using standard conditions.

Transcriptomic studies of individual and pooled perturbation experiments will be paired with phenotypic interrogation of neuronal maturation to systematically evaluate morphological and functional properties of these neurons.

### Example 5. Modeling TDP43 Pathology in ALS Using iPSC-Derived Motor Neurons

Many mutations leading to familial ALS result in accumulation of misfolded proteins, ER stress and motor neuron degeneration. While these genes are expressed already in embryonic motor neurons, and many at a higher level compared to adult motor neurons, only mature motor neurons show signs of protein dyshomeostasis. Similarly, motor neurons derived from ALS patient-derived iPSCs do not show overt signs of protein pathology.

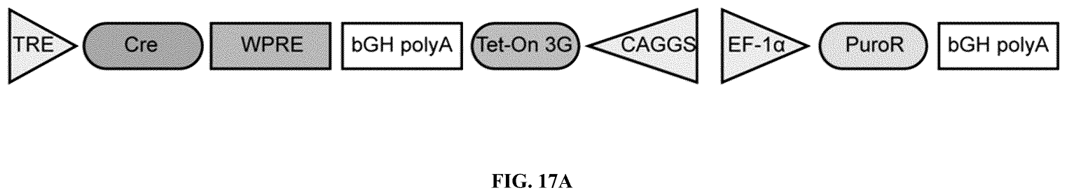

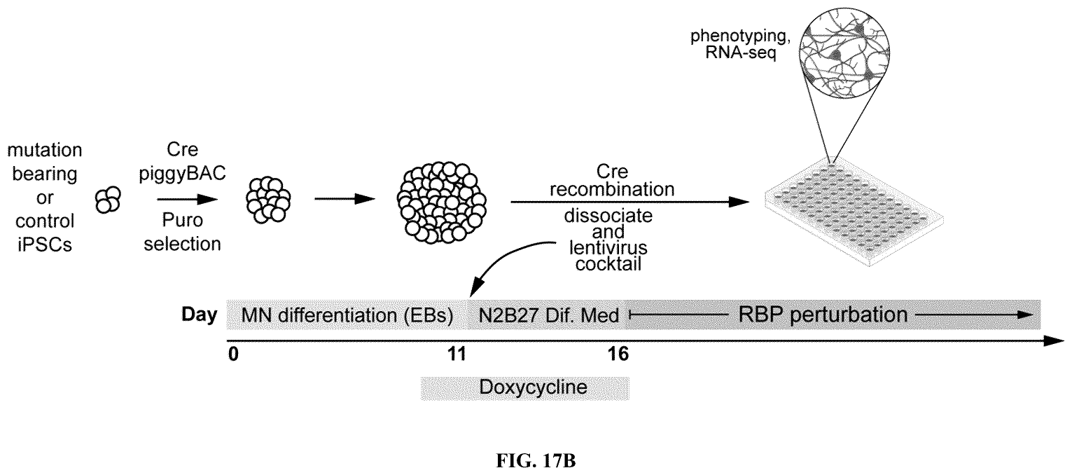

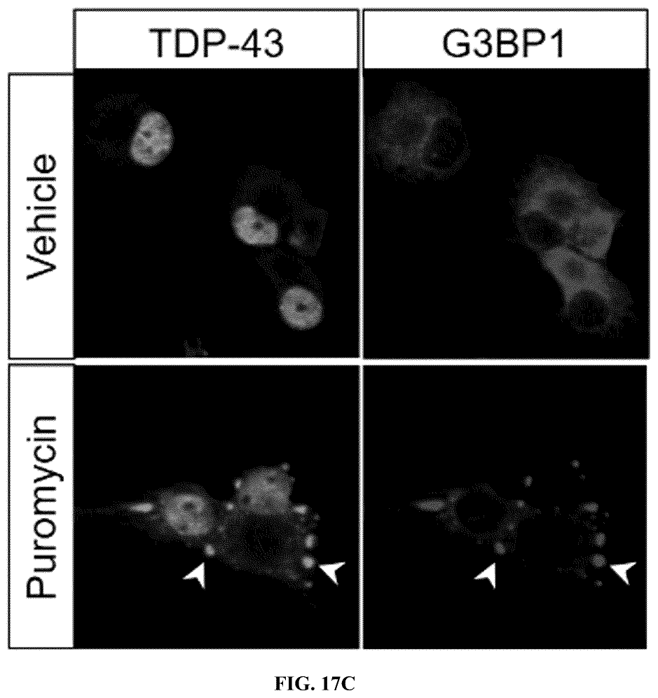

To evaluate whether motor neurons matured by splicing reprogramming become more susceptible to ALS-causing mutations, a doxycycline inducible Cre transgene will be introduced into two iPSC lines that were derived by the Columbia Stem Cell core from ALS patients carrying mutations in TARDBP gene encoding TDP-43, namely Q343R within the amyloidogenic core region of TDP-43 and second one carrying mutation A382T. These mutations increase propensity of TDP43 to form phosphorylated-TDP43 (p-TDP43) aggregates in ALS patients, but this phenotype cannot be recapitulated in iPSC-derived motor neurons using standard culture conditions. A tetracycline inducible Cre will be inserted into each of the iPSC lines using a piggyBAC construct (FIGS. 17A and 17B). Motor neurons differentiated from these lines will be transduced with the optimized combination of RBPs or control lentivirus carrying FLEx-GFP, plated in 96-well Greiner plates at low density (˜100 motor neurons per well) and cultured for varying number of days. Motor neuron survival and morphology will be evaluated by live imaging of calcein labeled cells, followed by MetaMorph® analysis, as described above. Plates will then be fixed and immunostained for ISL1 (motor neuron marker) and p-TDP43 to quantify the numbers and size of p-TDP43 aggregates in each motor neuron (FIG. 17C).

### REFERENCES CITED AND INCORPORATED BY REFERENCE

1. Agostini, M. et al. Metabolic reprogramming during neuronal differentiation. Cell Death Differ 23, 1502-1514 (2016).
2. Amoroso, M. W. et al. Accelerated high-yield generation of limb-innervating motor neurons from human stem cells. J Neurosci 33, 574-586 (2013).
3. Andreadis, A. Tau gene alternative splicing: expression patterns, regulation and modulation of function in normal brain and neurodegenerative diseases. Biochim Biophys Acta 1739, 91-103 (2005).
4. Arbab, M., S. Baars, and N. Geijsen, Modeling motor neuron disease: the matter of time. Trends Neurosci, 2014. 37(11): p. 642-52.
5. Baker, B. S. Sex in flies: the splice of life. Nature 340, 521-524 (1989).
6. Berry, B. J., et al., Advances and Current Challenges Associated with the Use of Human Induced Pluripotent Stem Cells in Modeling Neurodegenerative Disease. Cells Tissues Organs, 2018. 205(5-6): p. 331-349.
7. Boillee, S., Vande Velde, C. & Cleveland, D. W. ALS: a disease of motor neurons and their nonneuronal neighbors. Neuron 52, 39-59 (2006).
8. Brown, A. L., et al., TDP-43 loss and ALS-risk SNPs drive mis-splicing and depletion of UNC13A. Nature, 2022. 603(7899): p. 131-137.
9. Caillet-Boudin, M. L., et al., Regulation of human MAPT gene expression. Mol Neurodegener, 2015. 10: p. 28.
10. Capano, L. S. et al. Recapitulation of endogenous 4R tau expression and formation of insoluble tau in directly reprogrammed human neurons. Cell Stem Cell 29, 918-932 e918 (2022).
11. Chang, C. W., Shao, E. & Mucke, L. Tau: Enabler of diverse brain disorders and target of rapidly evolving therapeutic strategies. Science 371 (2021).
12. Charizanis, K. et al. Muscleblind-like 2-mediated alternative splicing in the developing brain and dysregulation in myotonic dystrophy. Neuron 75, 437-450 (2012).
13. Ciceri, G. et al. An epigenetic barrier sets the timing of human neuronal maturation. Nature 626, 881-890 (2024).
14. Dawson, T. M., T. E. Golde, and C. Lagier-Tourenne, Animal models of neurodegenerative diseases. Nat Neurosci, 2018. 21(10): p. 1370-1379.
15. Dewey, C. M. et al. TDP-43 aggregation in neurodegeneration: are stress granules the key? Brain Res 1462, 16-25 (2012).
16. Dillman, A. A., et al., mRNA expression, splicing and editing in the embryonic and adult mouse cerebral cortex. Nat Neurosci, 2013. 16(4): p. 499-506.
17. Ebisuya, M. & Briscoe, J. What does time mean in development? Development 145 (2018).
18. Flitsch, L. J., K. E. Laupman, and O. Brustle, Transcription Factor-Based Fate Specification and Forward Programming for Neural Regeneration. Front Cell Neurosci, 2020. 14: p. 121.
19. Gallegos, D. A., Chan, U., Chen, L. F. & West, A. E. Chromatin Regulation of Neuronal Maturation and Plasticity. Trends Neurosci 41, 311-324 (2018).
20. Gao, Q. S. et al. Complex regulation of tau exon 10, whose missplicing causes frontotemporal dementia. J Neurochem 74, 490-500 (2000).
21. Garcia-Diaz, A. et al. Standardized reporter systems for purification and imaging of human pluripotent stem cell-derived motor neurons and other cholinergic cells. Neuroscience (2020).
22. Gitler, A. D., P. Dhillon, and J. Shorter, Neurodegenerative disease: models, mechanisms, and a new hope. Dis Model Mech, 2017. 10(5): p. 499-502.
23. Goedert, M. & Jakes, R. Expression of separate isoforms of human tau protein: correlation with the tau pattern in brain and effects on tubulin polymerization. EMBO J 9, 4225-4230 (1990).
24. Goedert, M., Spillantini, M. G., Potier, M. C., Ulrich, J. & Crowther, R. A. Cloning and sequencing of the cDNA encoding an isoform of microtubule-associated protein tau containing four tandem repeats: differential expression of tau protein mRNAs in human brain. EMBO J 8, 393-399 (1989).
25. Goodwin, M. et al. MBNL sequestration by toxic RNAs and RNA misprocessing in the myotonic dystrophy brain. Cell Rep 12, 1159-1168 (2015).
26. Han, H., et al., MBNL proteins repress ES-cell-specific alternative splicing and reprogramming. Nature, 2013. 498(7453): p. 241-5.
27. Hao, Y. et al. Dictionary learning for integrative, multimodal and scalable single-cell analysis. Nat Biotechnol 42, 293-304 (2024).
28. Heemels, M. T., Neurodegenerative diseases. Nature, 2016. 539(7628): p. 179.
29. Hernandez, F., et al., Differences Between Human and Murine Tau at the N-terminal End. Front Aging Neurosci, 2020. 12: p. 11.
30. Ho, R. et al. ALS disrupts spinal motor neuron maturation and aging pathways within gene co-expression networks. Nat Neurosci 19, 1256-1267 (2016).
31. Hong, M., et al., Mutation-specific functional impairments in distinct tau isoforms of hereditary FTDP-17. Science, 1998. 282(5395): p. 1914-7.
32. Huh, C. J. et al. Maintenance of age in human neurons generated by microRNA-based neuronal conversion of fibroblasts. Elife 5 (2016).
33. Hutton, M. et al. Association of missense and 5′-splice-site mutations in tau with the inherited dementia FTDP-17. Nature 393, 702-705 (1998).
34. Iacovino, M., et al., Inducible cassette exchange: a rapid and efficient system enabling conditional gene expression in embryonic stem and primary cells. Stem Cells, 2011. 29(10): p. 1580-8.
35. Iijima, T. et al. SAM68 regulates neuronal activity-dependent alternative splicing of neurexin-1. Cell 147, 1601-1614 (2011).
36. Jacko, M. et al. Rbfox splicing factors promote neuronal maturation and axon initial segment assembly. Neuron 97, p 853-868.e856 (2018).
37. Jessell, T. M. Neuronal specification in the spinal cord: inductive signals and transcriptional codes. Nat Rev Genet 1, 20-29 (2000).
38. Karch, C. M., et al., A Comprehensive Resource for Induced Pluripotent Stem Cells from Patients with Primary Tauopathies. Stem Cell Reports, 2019. 13 (5): p. 939-955.
39. Kino, Y., et al., Nuclear localization of MBNL1: splicing-mediated autoregulation and repression of repeat-derived aberrant proteins. Hum Mol Genet, 2015. 24(3): p. 740-56.
40. Kosik, K. S., Orecchio, L. D., Bakalis, S. & Neve, R. L. Developmentally regulated expression of specific tau sequences. Neuron 2, 1389-1397 (1989).
41. Lee, J. A. et al. Cytoplasmic Rbfox1 regulates the expression of synaptic and autism-related genes. Neuron 89, 113-128 (2016).
42. Lee, K. Y., et al., Deprivation of Muscleblind-Like Proteins Causes Deficits in Cortical Neuron Distribution and Morphological Changes in Dendritic Spines and Postsynaptic Densities. Front Neuroanat, 2019. 13: p. 75.
43. Limone, F., J. R. Klim, and D. A. Mordes, Pluripotent stem cell strategies for rebuilding the human brain. Front Aging Neurosci, 2022. 14: p. 1017299.
44. Lin, L., Zhang, M., Stoilov, P., Chen, L. & Zheng, S. Developmental attenuation of neuronal apoptosis by neural-specific splicing of Bak1 microexon. Neuron 107, 1180-1196 e1188 (2020).
45. Lindholm, D., Wootz, H. & Korhonen, L. ER stress and neurodegenerative diseases. Cell Death Differ 13, 385-392 (2006).
46. Lister, R. & Mukamel, E. A. Turning over DNA methylation in the mind. Front Neurosci 9, 252 (2015).
47. Lister, R. et al. Global epigenomic reconfiguration during mammalian brain development. Science 341, 1237905 (2013).
48. Liu, E. Y., C. P. Cali, and E. B. Lee, RNA metabolism in neurodegenerative disease. Dis Model Mech, 2017. 10(5): p. 509-518.
49. Love, M. I., W. Huber, and S. Anders, Moderated estimation of fold change and dispersion for RNA-seq data with DESeq2. Genome Biol, 2014. 15(12): p. 550.
50. Ma, X. R., et al., TDP-43 represses cryptic exon inclusion in the FTD-ALS gene UNC13A. Nature, 2022. 603(7899): p. 124-130.
51. Maimon, R. et al. Therapeutically viable generation of neurons with antisense oligonucleotide suppression of PTB. Nat Neurosci 24, 1089-1099 (2021).
52. Marasco, L. E. and A. R. Kornblihtt, The physiology of alternative splicing. Nat Rev Mol Cell Biol, 2022.
53. Mazin, P. V., et al., Alternative splicing during mammalian organ development. Nat Genet, 2021. 53(6): p. 925-934.
54. Mazzoni, E. O. et al. Synergistic binding of transcription factors to cell-specific enhancers programs motor neuron identity. Nat Neurosci 16, 1219-1227 (2013).
55. McAllister, A. K., L. C. Katz, and D. C. Lo, Neurotrophin regulation of cortical dendritic growth requires activity. Neuron, 1996. 17(6): p. 1057-64.
56. Mead, R. J., Shan, N., Reiser, H. J., Marshall, F. & Shaw, P. J. Amyotrophic lateral sclerosis: a neurodegenerative disorder poised for successful therapeutic translation. Nat Rev Drug Discov 22, 185-212 (2023).
57. Meyer, B. J. Sex in the worm: counting and compensating X-chromosome dose. Trends Genet. 16, 247-253 (2000).
58. Miguel, L., et al., Detection of all adult Tau isoforms in a 3D culture model of iPSC-derived neurons. Stem Cell Res, 2019. 40: p. 101541.
59. Miles, G. B. et al. Functional properties of motoneurons derived from mouse embryonic stem cells. J Neurosci 24, 7848-7858 (2004).
60. Nehme, R., et al., Combining NGN2 Programming with Developmental Patterning Generates Human Excitatory Neurons with NMDAR-Mediated Synaptic Transmission. Cell Rep, 2018. 23(8): p. 2509-2523.
61. Nord, A. S. et al. Rapid and pervasive changes in genome-wide enhancer usage during mammalian development. Cell 155, 1521-1531 (2013).
62. Pan, Q., Shai, O., Lee, L. J., Frey, B. J. & Blencowe, B. J. Deep surveying of alternative splicing complexity in the human transcriptome by high-throughput sequencing. Nature Genet 40, 1413-1415 (2008).
63. Patel, T. et al. Transcriptional dynamics of murine motor neuron maturation in vivo and in vitro. Nat Commun 13, 5427 (2022).
64. Peljto, M., Dasen, J. S., Mazzoni, E. O., Jessell, T. M. & Wichterle, H. Functional diversity of ESC-derived motor neuron subtypes revealed through intraspinal transplantation. Cell Stem Cell 7, 355-366 (2010).
65. Porter, R. S., F. Jaamour, and S. Iwase, Neuron-specific alternative splicing of transcriptional machineries: Implications for neurodevelopmental disorders. Mol Cell Neurosci, 2018. 87: p. 35-45.
66. Raj, B. & Blencowe, B. J. Alternative splicing in the mammalian nervous system: recent insights into mechanisms and functional roles. Neuron 87, 14-27 (2015).
67. Rimington, R. P., et al., Bioengineered model of the human motor unit with physiologically functional neuromuscular junctions. Sci Rep, 2021. 11(1): p. 11695.
68. Sandoe, J. and K. Eggan, Opportunities and challenges of pluripotent stem cell neurodegenerative disease models. Nat Neurosci, 2013. 16(7): p. 780-9.
69. Schaler, A. W., et al., PAC1 receptor-mediated clearance of tau in postsynaptic compartments attenuates tau pathology in mouse brain. Sci Transl Med, 2021. 13 (595).
70. Schoch, K. M., et al., Increased 4R-Tau Induces Pathological Changes in a Human-Tau Mouse Model. Neuron, 2016. 90 (5): p. 941-7.
71. Shah, A., Qian, Y., Weyn-Vanhentenryck, S. M. & Zhang, C. CLIP Tool Kit (CTK): a flexible and robust pipeline to analyze CLIP sequencing data. Bioinformatics 33, 566-567 (2017).
72. Smith, B. J. & Carregari, V. C. Post-Translational Modifications During Brain Development. Adv Exp Med Biol 1382, 29-38 (2022).
73. Spillantini, M. G. et al. Mutation in the tau gene in familial multiple system tauopathy with presenile dementia. Proc Natl Acad Sci USA 95, 7737-7741 (1998).
74. Sposito, T. et al. Developmental regulation of tau splicing is disrupted in stem cell-derived neurons from frontotemporal dementia patients with the 10+16 splice-site mutation in MAPT. Hum Mol Genet 24, 5260-5269 (2015).
75. Sternfeld, M. J. et al. Speed and segmentation control mechanisms characterized in rhythmically-active circuits created from spinal neurons produced from genetically-tagged embryonic stem cells. Elife 6 (2017).
76. Strang, K. H., T. E. Golde, and B. I. Giasson, MAPT mutations, tauopathy, and mechanisms of neurodegeneration. Lab Invest, 2019. 99(7): p. 912-928.
77. Stroud, H. et al. An activity-mediated transition in transcription in early postnatal neurons. Neuron 107, 874-890 e878 (2020).
78. Stuart, T., Srivastava, A., Madad, S., Lareau, C. A. & Satija, R. Single-cell chromatin state analysis with Signac. Nat Methods 18, 1333-1341 (2021).
79. Suresh, S. N., et al., Neurodegenerative diseases: model organisms, pathology and autophagy. J Genet, 2018. 97 (3): p. 679-701.
80. Takazawa, T. et al. Maturation of spinal motor neurons derived from human embryonic stem cells. PLoS One 7, e40154 (2012).
81. Tan, L. et al. Changes in genome architecture and transcriptional dynamics progress independently of sensory experience during post-natal brain development. Cell 184, 741-758 e717 (2021).
82. Telonis-Scott, M., et al., Sex-specific splicing in Drosophila: widespread occurrence, tissue specificity and evolutionary conservation. Genetics, 2009. 181(2): p. 421-34.
83. Thams, S. et al. A stem cell-based screening platform identifies compounds that desensitize motor neurons to endoplasmic reticulum stress. Molecular therapy: the journal of the American Society of Gene Therapy 27, 87-101 (2019).
84. The Alzheimer's Association 2023 Alzheimer's disease facts and figures. Alzheimers Dement 19, 1598-1695 (2023).
85. Toma, K., Wang, T. C. & Hanashima, C. Encoding and decoding time in neural development. Dev Growth Differ 58, 59-72 (2016).
86. Trabzuni, D., et al., Widespread sex differences in gene expression and splicing in the adult human brain. Nat Commun, 2013. 4: p. 2771.
87. Traunmuller, L., Gomez, A. M., Nguyen, T. M. & Scheiffele, P. Control of neuronal synapse specification by a highly dedicated alternative splicing program. Science 352, 982-986 (2016).
88. Ule, J. and B. J. Blencowe, Alternative Splicing Regulatory Networks: Functions, Mechanisms, and Evolution. Mol Cell, 2019. 76(2): p. 329-345.
89. Ullian, E. M., et al., Control of synapse number by glia. Science, 2001. 291(5504): p. 657-61.
90. Vaquero-Garcia, J., et al., A new view of transcriptome complexity and regulation through the lens of local splicing variations. Elife, 2016. 5: p. e11752.
91. Verheyen, A., et al., Using Human iPSC-Derived Neurons to Model TAU Aggregation. PLoS One, 2015. 10(12): p. e0146127.
92. Vickers, T. A., et al., Fully modified 2′ MOE oligonucleotides redirect polyadenylation. Nucleic Acids Res, 2001. 29(6): p. 1293-9.
93. Vierbuchen, T., et al., Direct conversion of fibroblasts to functional neurons by defined factors. Nature, 2010. 463(7284): p. 1035-41.
94. Vuong, C. K. et al. Rbfox1 regulates synaptic transmission through the inhibitory neuron-specific vSNARE Vamp1. Neuron 98, 127-141 e127 (2018).
95. Vuong, C. K., D. L. Black, and S. Zheng, The neurogenetics of alternative splicing. Nat Rev Neurosci, 2016. 17(5): p. 265-81.
96. Wainger, B. J. et al. Intrinsic membrane hyperexcitability of amyotrophic lateral sclerosis patient-derived motor neurons. Cell Rep 7, 1-11 (2014).
97. Wallace, J. L. & Pollen, A. A. Human neuronal maturation comes of age: cellular mechanisms and species differences. Nat Rev Neurosci 25, 7-29 (2024).
98. Wang, E. T. et al. Alternative isoform regulation in human tissue transcriptomes. Nature 456, 470-476 (2008).
99. Wang, E. T. et al. Transcriptome-wide regulation of pre-mRNA splicing and mRNA localization by muscleblind proteins. Cell 150, 710-724 (2012).
100. Wang, L. H., et al., Calpain-2 Mediates MBNL2 Degradation and a Developmental RNA Processing Program in Neurodegeneration. J Neurosci, 2022. 42(25): p. 5102-5114.
101. Wang, Y. & Mandelkow, E. Tau in physiology and pathology. Nat Rev Neurosci 17, 5-21 (2016).
102. Weyn-Vanhentenryck, S. et al. HITS-CLIP and integrative modeling define the Rbfox splicing-regulatory network linked to brain development and autism. Cell Rep. 6, 1139-1152 (2014).
103. Weyn-Vanhentenryck, S. M. et al. Precise temporal regulation of alternative splicing during neural development. Nat Commun, 2189 (2018).
104. Wichterle, H., et al., Directed differentiation of embryonic stem cells into motor neurons. Cell, 2002. 110(3): p. 385-97.
105. Wilkinson, M. E., C. Charenton, and K. Nagai, RNA Splicing by the Spliceosome. Annu Rev Biochem, 2020. 89: p. 359-388.
106. Xue, Y. et al. Direct conversion of fibroblasts to neurons by reprogramming PTB-regulated microRNA circuits. Cell 152, 82-96 (2013).
107. Yan, Q. et al. Systematic discovery of regulated and conserved alternative exons in the mammalian brain reveals NMD modulating chromatin regulators. Proc Natl Acad Sci USA 112, 3445-3350 (2015).
108. Yang, Y. M. et al. A small molecule screen in stem-cell-derived motor neurons identifies a kinase inhibitor as a candidate therapeutic for ALS. Cell Stem Cell 12, 713-726 (2013).
109. Yoo, A. S., Staahl, B. T., Chen, L. & Crabtree, G. R. MicroRNA-mediated switching of chromatin-remodelling complexes in neural development. Nature 460, 642-646 (2009).
110. Zakrzewski, W., Dobrzynski, M., Szymonowicz, M. & Rybak, Z. Stem cells: past, present, and future. Stem Cell Res Ther 10, 68 (2019).
111. Zhang, C. et al. Integrative modeling defines the Nova splicing-regulatory network and its combinatorial controls. Science 329, 439-443 (2010).
112. Zhang, Y. et al. An RNA-sequencing transcriptome and splicing database of glia, neurons, and vascular cells of the cerebral cortex. J Neurosci 34, 11929-11947 (2014).
113. Zheng, S. et al. PSD-95 is post-transcriptionally repressed during early neural development by PTBP1 and PTBP2. Nat Neurosci 15, 381-388, S381 (2012).
114. Zheng, X. et al. Metabolic reprogramming during neuronal differentiation from aerobic glycolysis to neuronal oxidative phosphorylation. Elife 5 (2016).
115. Zhong, Q., et al., Tau isoform composition influences rate and extent of filament formation. J Biol Chem, 2012. 287(24): p. 20711-9.

I/We claim:

1. A method of generating mature neurons from differentiated stem cells comprising:
differentiating pluripotent stem cells into neurons; and
overexpressing Mbnl2 in the differentiated pluripotent stem cells.

2. The method of claim 1, wherein Mbnl2 is overexpressed in the differentiated pluripotent stem cells by nucleofecting the differentiating pluripotent stem cell with a lentivirus construct for overexpressing Mbnl2.

3. The method of claim 2, wherein the lentivirus construct for overexpressing Mbnl2 has a sequence set forth in SEQ ID NO. 2 or SEQ ID NO. 3.

4. The method of claim 3, wherein the pluripotent stem cells expresses Cre.

5. The method of claim 1, further comprising overexpressing Rbfox1 in the pluripotent stem cells.

6. The method of claim 1, further comprising reducing expression of PTBP1/2 in the pluripotent stem cells.

7. The method of claim 1, wherein the pluripotent stem cells conditionally express Mbnl2, the step of overexpressing Mbnl2 in the differentiated pluripotent stem cells comprises inducing expression of Mbnl2 during the step of differentiating pluripotent stem cells.

8. The method of claim 7, wherein Mbnl2 is conditionally expressed in the pluripotent stem cells by transfecting the pluripotent stem cell with a conditional expression construct for overexpressing Mbnl2.

9. The method of claim 7, further comprising overexpressing Rbfox1 in the pluripotent stem cells.

10. The method of claim 7, further comprising reducing expression of PTBP1/2 in the pluripotent stem cells.

11. A method of accelerating maturation of a neuron, the method comprising:
overexpressing Mbnl2 in the neuron;
overexpressing Rbfox1 in the neuron; and
reducing expression of PTBP1/2 in the neuron,
wherein expression of Mbnl2, Rbfox1, and PTBP1/2 are simultaneously modulated in the neuron.

12. The method of claim 11, wherein Mbnl2 is overexpressed in the neuron by nucleofecting the neuron with a lentivirus construct for overexpressing Mbnl2.

13. The method of claim 12, wherein the lentivirus construct for overexpressing Mbnl2 has a sequence set forth in SEQ ID NO. 2 or SEQ ID NO. 3.

14. The method of claim 11, wherein the neuron expresses Cre.

15. The method of claim 12, wherein Mbnl2 is overexpressed in the pluripotent stem cells by transfecting the pluripotent stem cell with a conditional expression construct for overexpressing Mbnl2.

16. The method of claim 12, wherein the neuron is differentiated from a culture of pluripotent stem cells.

17. The method of claim 16, wherein the method generates a tau pathology model.

18. The method of claim 12, wherein the neuron is a motor neuron.

19. A composition for inducing maturation of a neuron comprising:
a construct for overexpressing Mbnl2; and
a construct for reducing expression of PTBP1/2.

20. The composition of claim 19, wherein the construct for overexpressing Mbnl2 further overexpresses Rbfox1.


SEQUENCE LISTING (3 sequences)

SEQ ID NO: 1 - DNA, 11166 bp, synthetic construct
  source 1..11166: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaagcggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtaccgagctcgaattgggatccaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattccggaataacttcgtataggatactttatacgaagttatgcagaatggtagctggattgtagctgctattagcaatatgaaacctcttaataacttcgtatagcatacattatacgaagttattggcgcgccgccacccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgccttaattaagcttgtgccccagtttgctagggaggtcgcagtatctggccactgccacctcgtgctgctcgacgtaggtctcgttgttggcctccttgattctttccagtctgtagtccacatagtagacgccaggcatcttgaggttcttagcgggtttcttggatctatatgtggtcttggcgtttgcgatcagatggctcccgcccacgagcttcagggccatgtcgtttctgccttccaggccgccgtcagcggggtacagcgtctcggtgaaggcctcccagccgagtgttttcttctgcatcacagggccgttggatgtgaagttcacccctctgatcttgacgttgtagatgaggcagccgtcctggaggctggtgtcctgggtagcggtcagcacgcccccgtcttcgtatgtggtgactctctcccatgtgaagccctcagggaaggactgcttgaagaagtcggggatgccctgggtgtggttgatgaaggtcttgctgccgtagaggaagctagtagccaggatgtcgaaggcgaaggggagagggccgccctcgaccaccttgattctcatggtctgggtgccctcgtagggcttgccttcgccctcggatgtgcacttgaagtgatggttgtccacggtgccctccatgtacagcttcatgtgcatgttctccttaatcagctcgctcatggtggcgctagcataacttcgtataaagtatcctatacgaagttatttgccttaacccagaaattatcactgttattctttagaatggtgcaaagaataacttcgtataatgtatgctatacgaagttatgaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccccgcccccatcgctgcacaaaataattaaaaaataaataaatacaaaattgggggtggggagggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcgacatcgataaaattttgaattttgtaatttgtttttgtaattctttagtttgtatgtctgttgctattatgtctactattctttcccctgcactgtaccccccaatccccccttttcttttaaaagttgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 2 - DNA, 11621 bp, synthetic construct
  source 1..11621: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaagcggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtaccgagctcgaattgggatccaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattccggaataacttcgtataggatactttatacgaagttatgcagaatggtagctggattgtagctgctattagcaatatgaaacctcttaataacttcgtatagcatacattatacgaagttattggcgcgccgccaccaccatggctagcgactataaagatgatgatgataaggattacaaggacgacgacgacaaggactacaaagacgatgacgataaagctagcgagaacctctacttccaatcgaccggtgccttgaacgttgcccccgtgagagacacaaagtggctgacgctggaggtctgcagacagtaccagagaggaacgtgctcacgctccgacgaagaatgcaagtttgctcacccccccaaaagttgccaggttgaaaatggaagagtaattgcctgctttgattccctcaagggccgctgttcaagagagaactgcaaatatcttcatcctccgacacacttaaaaacccagctagagattaatgggaggaacaatttgatccagcaaaaaactgcagcagcgatgcttgcccagcagatgcaatttatgtttccaggaacgccgctccatcctgtgcccacttttcctgtaggtcccaccatagggacaaatgcggctattagctttgctccttacttagcgcctgtcacccctggagttgggttagtcccaacagaggttctacccactacaccggtcattgttcccggaagtccaccggtcactgtcccgggctcaactgcaactcagaaacttctcaggactgataaactggaggtatgcagggagttccagcgaggaaactgtgcccggggagagacagactgccgctttgcacacccggcagacagcaccatgatcgacacaaacgacaacaccgtaaccgtttgtatggattacataaaggggcgttgcatgagggagaaatgcaaatattttcaccctcctgcacacttgcaggccaaaatcaaagctgcgcagcaccaagccaaccaggccgcggtggccgcccaggcagccgcggccgcggccacagtcatggccttccctccgggtgctcttcatcccttaccaaagagacaagcacttgaaaaaagcaacggggccagcacggtcttcaaccccagcgtcttgcactaccagcaggctctgaccagtgcgcagctgcagcagcacacggcgttcatccccacagtacccatgatgcacagcgctacgtccgccactgtctctgcagcaacaactcctgcaacaagtgtccccttcgcagcaacagccacagccaatcagataattctgaaacttaagtaactcgaggccgcagccttaattaagcttgtgccccagtttgctagggaggtcgcagtatctggccactgccacctcgtgctgctcgacgtaggtctcgttgttggcctccttgattctttccagtctgtagtccacatagtagacgccaggcatcttgaggttcttagcgggtttcttggatctatatgtggtcttggcgtttgcgatcagatggctcccgcccacgagcttcagggccatgtcgtttctgccttccaggccgccgtcagcggggtacagcgtctcggtgaaggcctcccagccgagtgttttcttctgcatcacagggccgttggatgtgaagttcacccctctgatcttgacgttgtagatgaggcagccgtcctggaggctggtgtcctgggtagcggtcagcacgcccccgtcttcgtatgtggtgactctctcccatgtgaagccctcagggaaggactgcttgaagaagtcggggatgccctgggtgtggttgatgaaggtcttgctgccgtagaggaagctagtagccaggatgtcgaaggcgaaggggagagggccgccctcgaccaccttgattctcatggtctgggtgccctcgtagggcttgccttcgccctcggatgtgcacttgaagtgatggttgtccacggtgccctccatgtacagcttcatgtgcatgttctccttaatcagctcgctcatggtggcgctagcataacttcgtataaagtatcctatacgaagttatttgccttaacccagaaattatcactgttattctttagaatggtgcaaagaataacttcgtataatgtatgctatacgaagttatgaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccccgcccccatcgctgcacaaaataattaaaaaataaataaatacaaaattgggggtggggagggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcgacatcgataaaattttgaattttgtaatttgtttttgtaattctttagtttgtatgtctgttgctattatgtctactattctttcccctgcactgtaccccccaatccccccttttcttttaaaagttgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 3 - DNA, 11675 bp, synthetic construct
  source 1..11675: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaagcggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtaccgagctcgaattgggatccaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattccggaataacttcgtataggatactttatacgaagttatgcagaatggtagctggattgtagctgctattagcaatatgaaacctcttaataacttcgtatagcatacattatacgaagttattggcgcgccgccaccaccatggctagcgactataaagatgatgatgataaggattacaaggacgacgacgacaaggactacaaagacgatgacgataaagctagcgagaacctctacttccaatcgaccggtgccttgaacgttgcccccgtgagagacacaaagtggctgacgctggaggtctgcagacagtaccagagaggaacgtgctcacgctccgacgaagaatgcaagtttgctcacccccccaaaagttgccaggttgaaaatggaagagtaattgcctgctttgattccctcaagggccgctgttcaagagagaactgcaaatatcttcatcctccgacacacttaaaaacccagctagagattaatgggaggaacaatttgatccagcaaaaaactgcagcagcgatgcttgcccagcagatgcaatttatgtttccaggaacgccgctccatcctgtgcccacttttcctgtaggtcccaccatagggacaaatgcggctattagctttgctccttacttagcgcctgtcacccctggagttgggttagtcccaacagaggttctacccactacaccggtcattgttcccggaagtccaccggtcactgtcccgggctcaactgcaactcagaaacttctcaggactgataaactggaggtatgcagggagttccagcgaggaaactgtgcccggggagagacagactgccgctttgcacacccggcagacagcaccatgatcgacacaaacgacaacaccgtaaccgtttgtatggattacataaaggggcgttgcatgagggagaaatgcaaatattttcaccctcctgcacacttgcaggccaaaatcaaagctgcgcagcaccaagccaaccaggccgcggtggccgcccaggcagccgcggccgcggccacagtcatgactcagtcgactgccaaagcactgaagcgacctctcgaagcaactgtagacctggccttccctccgggtgctcttcatcccttaccaaagagacaagcacttgaaaaaagcaacggggccagcacggtcttcaaccccagcgtcttgcactaccagcaggctctgaccagtgcgcagctgcagcagcacacggcgttcatccccacagtacccatgatgcacagcgctacgtccgccactgtctctgcagcaacaactcctgcaacaagtgtccccttcgcagcaacagccacagccaatcagataattctgaaacttaagtaactcgaggccgcagccttaattaagcttgtgccccagtttgctagggaggtcgcagtatctggccactgccacctcgtgctgctcgacgtaggtctcgttgttggcctccttgattctttccagtctgtagtccacatagtagacgccaggcatcttgaggttcttagcgggtttcttggatctatatgtggtcttggcgtttgcgatcagatggctcccgcccacgagcttcagggccatgtcgtttctgccttccaggccgccgtcagcggggtacagcgtctcggtgaaggcctcccagccgagtgttttcttctgcatcacagggccgttggatgtgaagttcacccctctgatcttgacgttgtagatgaggcagccgtcctggaggctggtgtcctgggtagcggtcagcacgcccccgtcttcgtatgtggtgactctctcccatgtgaagccctcagggaaggactgcttgaagaagtcggggatgccctgggtgtggttgatgaaggtcttgctgccgtagaggaagctagtagccaggatgtcgaaggcgaaggggagagggccgccctcgaccaccttgattctcatggtctgggtgccctcgtagggcttgccttcgccctcggatgtgcacttgaagtgatggttgtccacggtgccctccatgtacagcttcatgtgcatgttctccttaatcagctcgctcatggtggcgctagcataacttcgtataaagtatcctatacgaagttatttgccttaacccagaaattatcactgttattctttagaatggtgcaaagaataacttcgtataatgtatgctatacgaagttatgaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccccgcccccatcgctgcacaaaataattaaaaaataaataaatacaaaattgggggtggggagggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcgacatcgataaaattttgaattttgtaatttgtttttgtaattctttagtttgtatgtctgttgctattatgtctactattctttcccctgcactgtaccccccaatccccccttttcttttaaaagttgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc